(19) United States
(12) Patent Application Publication
(10) Pub. No.: US 2023/0233661 A1
(43) Pub. Date: Jul. 27, 2023

(54) VACCINE COMBINATION AGAINST REPIRATORY SYNCYTIAL VIRUS INFECTION

(71) Applicant: Janssen Vaccines & Prevention B.V., Leiden (NL)

(72) Inventors: Benoit Christophe Stephan CALLENDRET, Den Haag (NL); Els DE PAEPE, Lokeren (BE); Christy Ann COMEAUX, Amsterdam (NL); Roland Christian ZAHN, Rijnsburg (NL); Esther Mathilde Eugene Wilhelmus HEIJNEN, 's Hertogenbosch (NL)

(21) Appl. No.: 18/003,547

(22) PCT Filed: Jun. 29, 2021
(86) PCT No.: PCT/EP2021/067776

§ 371 Date: Dec. 28, 2022

Related U.S. Application Data
(60) Provisional application No. 62/705,463, filed on Jun. 29, 2020.

(30) Foreign Application Priority Data
Jul. 23, 2020 (EP) 20187409.6

(51) Int. Cl. A61K 39/155; A61P 31/14; A61K 39/00

(52) U.S. Cl. CPC A61K 39/155; A61P 31/14; C12N 2760/18534; A61K 2039/53; C12N 2710/10043; A61K 2039/54; A61K 2039/545

(57) ABSTRACT

Methods of safely inducing a protective immune response against respiratory syncytial virus (RSV) and methods of preventing infection and/or replication of RSV in human subjects are described. The methods include administering to the subjects (a) an effective amount of an adenoviral vector encoding a recombinant RSV F protein that is stabilized in a pre-fusion conformation, and (b) an effective amount of an RSV F protein that is stabilized in a pre-fusion conformation.

### FIELD OF THE INVENTION

The present invention is in the field of medicine. In particular, embodiments of the invention relate to protective and immunogenic combinations of (a) a nucleic acid encoding a protein antigen of a Respiratory Syncytial Virus (RSV) and (b) a protein antigen of an RSV, and the use thereof for prophylactic treatment of RSV infection.

### BACKGROUND

Respiratory syncytial virus (RSV) is considered to be the most important cause of serious acute respiratory illness in infants and children under 5 years of age. Globally, RSV is responsible for an estimated 3.4 million hospitalizations annually. In the United States, RSV infection in children under 5 years of age is the cause of 57,000 to 175,000 hospitalizations, 500,000 emergency room visits, and approximately 500 deaths each year. In the US, 60% of infants are infected upon initial exposure to RSV, and nearly all children will have been infected with the virus by 2-3 years of age. Immunity to RSV is transient, and repeated infection occurs throughout life (Hall et al., J Infect Dis. 1991:163; 693-698). In children under 1 year of age, RSV is the most important cause of bronchiolitis, and RSV hospitalization is highest among children under 6 months of age (Centers for Disease Control and Prevention (CDC). Respiratory Syncytial Virus Infection (RSV)-Infection and Incidence. Almost all RSV-related deaths (99%) in children under 5 years of age occur in the developing world (Nair et al., Lancet. 2010:375; 1545-1555). Nevertheless, the disease burden due to RSV in developed countries is substantial, with RSV infection during childhood linked to the development of wheezing, airway hyperreactivity and asthma.

In addition to children, RSV is an important cause of respiratory infections in the elderly, immunocompromised, and those with underlying chronic cardio-pulmonary conditions (Falsey et al., N Engl J Med. 2005:352; 1749-1759). In long-term care facilities, RSV is estimated to infect 5-10% of the residents per year with significant rates of pneumonia (10 to 20%) and death (2 to 5%) (Falsey et al., Clin Microbiol Rev. 2000:13; 371-384). In one epidemiology study of RSV burden, it was estimated that 11,000 elderly persons die annually of RSV in the US (Thompson et al., JAMA. 2003:289; 179-186). These data support the importance of developing an effective vaccine for certain adult populations. Prophylaxis through passive immunization with a neutralizing monoclonal antibody against the RSV fusion (F) glycoprotein (Synagis® [palivizumab]) is available, but only indicated for premature infants (less than 29 weeks gestational age), children with severe cardio-pulmonary disease or those that are profoundly immunocompromised (American Academy of Pediatrics Committee on Infectious Diseases, American Academy of Pediatrics Bronchiolitis Guidelines Committee. Updated guidance for palivizumab prophylaxis among infants and young children at increased risk of hospitalization for respiratory syncytial virus infection. Pediatrics. 2014:134; 415-420). Synagis has been shown to reduce the risk of hospitalization by 55% (Prevention. Prevention of respiratory syncytial virus infections: indications for the use of palivizumab and update on the use of RSV-IGIV. American Academy of Pediatrics Committee on Infectious Diseases and Committee of Fetus and Newborn. Pediatrics. 1998:102; 1211-1216).

Despite the high disease burden and a strong interest in RSV vaccine development, no licensed vaccine is available for RSV. In the late 1960s, a series of studies were initiated to evaluate a formalin-inactivated RSV vaccine (FI-RSV) adjuvanted with alum, and the results of these studies had a major impact on the RSV vaccine field. Four studies were performed in parallel in children of different age groups with an FI-RSV vaccine delivered by intramuscular injection (Chin et al., Am J Epidemiol. 1969:89; 449-463; Fulginiti et al., Am J Epidemiol. 1969:89; 435-448; Kapikian et al., Am J Epidemiol. 1969:89; 405-421; Kim et al., Am J Epidemiol. 1969:89; 422-434). Eighty percent of the RSV-infected FI-RSV recipients required hospitalization and two children died during the next winter season (Chin et al., Am J Epidemiol. 1969:89; 449-463). Only 5% of the children in the RSV-infected control group required hospitalization. The mechanisms of the observed enhanced respiratory disease (ERD) among the FI-RSV recipients upon reinfection have been investigated and are believed to be the result of an aberrant immune response in the context of small bronchi present in that age group. Data obtained from analysis of patient samples and animal models suggest that FI-RSV ERD is characterized by low neutralizing antibody titers, the presence of low avidity non-neutralizing antibodies promoting immune complex deposition in the airways, reduced cytotoxic CD8+ T-cell priming shown to be important for viral clearance, and enhanced CD4+T helper type 2 (Th2)-skewed responses with evidence of eosinophilia (Beeler et al., Microb Pathog. 2013:55; 9-15; Connors et al., J Virol. 1992:66; 7444-7451; De Swart et al., J Virol. 2002:76; 11561-11569; Graham et al., J Immunol. 1993:151; 2032-2040; Kim et al., Pediatr Res. 1976:10; 75-78; Murphy et al., J Clin Microbiol. 1986:24; 197-202; Murphy et al., J Clin Microbiol. 1988:26; 1595-1597; Polack et al., J Exp Med. 2002:196; 859-865). It is believed that the chemical interaction of formalin and RSV protein antigens may be one of the mechanisms by which the FI-RSV vaccine promoted ERD upon subsequent RSV infection (Moghaddam et al., Nat Med. 2006:12; 905-907). For these reasons, formalin is no longer used in RSV vaccine development.

In addition to the FI-RSV vaccine, several live-attenuated and subunit RSV vaccines have been examined in animal models and human studies, but many have been inhibited by the inability to achieve the right balance of safety and immunogenicity/efficacy. Live-attenuated vaccines have been specifically challenged by difficulties related to over- and under-attenuation in infants (Belshe et al., J Infect Dis. 2004:190; 2096-2103; Karron et al., J Infect Dis. 2005:191; 1093-1104; Luongo et al., Vaccine. 2009:27; 5667-5676). With regard to subunit vaccines, the RSV fusion (F) and glycoprotein (G) proteins, which are both membrane proteins, are the only RSV proteins that induce neutralizing antibodies (Shay et al., JAMA. 1999:282; 1440-1446). Unlike the RSV G protein, the F protein is conserved between RSV strains. A variety of RSV F-subunit vaccines have been developed based on the known superior immunogenicity, protective immunity and the high degree of conservation of the F protein between RSV strains (Graham, Immunol Rev. 2011:239; 149-166). The proof-of-concept provided by the currently available anti-F protein neutralizing monoclonal antibody prophylaxis provides support for the idea that a vaccine inducing high levels of long-lasting neutralizing antibody may prevent RSV disease (Feltes et al., Pediatr Res. 2011:70; 186-191; Groothuis et al., J Infect Dis. 1998:177; 467-469; Groothuis et al., N Engl J Med. 1993:329; 1524-1530). Several studies have suggested that decreased protection against RSV in elderly could be attributed to an age-related decline in interferon gamma (IFNγ) production by peripheral blood mononuclear cells (PBMCs), a reduced ratio of CD8+ to CD4+ T cells, and reduced numbers of circulating RSV-specific CD8+ memory T cells (De Bree et al., J Infect Dis. 2005:191; 1710-1718; Lee et al., Mech Ageing Dev. 2005:126; 1223-1229; Looney et al., J Infect Dis. 2002:185; 682-685). High levels of serum neutralizing antibody are associated with less severe infections in elderly (Walsh and Falsey, J Infect Dis. 2004:190; 373-378). It has also been demonstrated that, following RSV infection in adults, serum antibody titers rise rapidly but then slowly return to pre-infection levels after 16 to 20 months (Falsey et al., J Med Virol. 2006:78; 1493-1497). With consideration given to the previously observed ERD in the FI-RSV vaccine studies in the 1960s, future vaccines should promote a strong antigen-specific CD8+ T-cell response and avoid a skewed Th2-type CD4+ T cell response (Graham, Immunol Rev. 2011:239; 149-166).

RSV F protein fuses the viral and host-cell membranes by irreversible protein refolding from the labile pre-fusion conformation to the stable post-fusion conformation. Structures of both conformations have been determined for RSV F (McLellan et al., Science 2013:342, 592-598; McLellan et al., Nat Struct Mol Biol 2010:17, 248-250; McLellan et al., Science 340, 2013:1113-1117; Swanson et al., Proceedings of the National Academy of Sciences of the United States of America 2011:108, 9619-9624), as well as for the fusion proteins from related paramyxoviruses, providing insight into the mechanism of this complex fusion machine. Like many other class I fusion proteins, RSV F undergoes proteolytic processing during maturation in the secretory pathway of infected cells. RSV F is synthesized as a single-chain inactive precursor (also called F0) that contains three subunits: F1, F2, and a 27-amino acid glycopeptide called pep27. This precursor must be cleaved by a furin-like protease to release pep27 and form the mature, fusion-competent protein (FIG. 1, RSV F mature processed). The C-terminal F1 subunit contains the transmembrane domain, two heptad repeats, and an N-terminal fusion peptide. Residues in the F2 subunit contribute to fusogenicity of the F protein and possibly the species specificity of RSV. In the mature processed protein, the F1 and F2 subunits are covalently associated via two disulfide bonds. Three F1-F2 protomers then associate via weak intermolecular interactions to form the trimeric, prefusion protein on the surface of the virion.

Most neutralizing antibodies in human sera are directed against the pre-fusion conformation, but due to its instability the pre-fusion conformation has a propensity to prematurely refold into the post-fusion conformation, both in solution and on the surface of the virions. Vaccines comprising RSV F proteins stabilized in a pre-fusion conformation, as well as vectors containing nucleic acid encoding RSV F proteins have been described. However, there is no report on the safety or efficacy of such proteins in humans. There is a currently still a high need for a safe and effective vaccine against RSV.

### SUMMARY OF THE INVENTION

The present application describes compositions and methods with increased immunogenic efficacy. More specifically, the application describes efficacious immunogenic combinations for concurrent administration, that elicit both potent B and T cell responses, thereby enhancing immunogenicity, and ultimately protection, against respiratory syncytial virus (RSV) infection.

In one general aspect, the present application describes a method for inducing a protective immune response against respiratory syncytial virus (RSV) infection in a human subject in need thereof, comprising administering to the subject an immunogenic combination of (a) an effective amount of a first immunogenic component, comprising an adenoviral vector comprising a nucleic acid encoding an RSV F protein that is stabilized in a pre-fusion conformation, preferably the effective amount of the first immunogenic component comprises from about 1×10¹⁰ to about 1×10¹² viral particles of the adenoviral vector per dose, and (b) an effective amount of a second immunogenic component, comprising a soluble RSV F protein that is stabilized in a pre-fusion conformation, preferably the effective amount of the second immunogenic component comprises about 30 ug to about 250 ug of the RSV F protein per dose.

In certain embodiments, the first and second immunogenic components are co-administered.

In certain embodiments, the first and second immunogenic components are formulated in different compositions, which are mixed prior to co-administration. The first and second immunogenic components may however also be co-formulated in one composition.

In certain preferred embodiments, the immunogenic components are administered intramuscularly, i.e. by intramuscular injection.

In certain embodiments, the adenoviral vector is replication-incompetent and has a deletion in at least one of the adenoviral early region 1 (E1 region) and the early region 3 (E3 region), or a deletion in both the E1 and the E3 region of the adenoviral genome.

In certain embodiments, the adenoviral vector is a replication-incompetent Ad26 adenoviral vector having a deletion of the E1 region and the E3 region.

In certain embodiments, the first immunogenic component is or comprises a replication-incompetent adenovirus serotype 26 (Ad26) containing a deoxyribonucleic acid (DNA) transgene that encodes the pre-F conformation-stabilized membrane-bound F protein derived from the RSV A2 strain, and the second immunogenic component is or comprises a recombinant, soluble, pre-F conformation-stabilized F protein derived from the RSV A2 strain.

According to the invention, the recombinant RSV F protein encoded by the adenoviral vector and the soluble RSV F protein have been stabilized in the pre-fusion conformation. Thus, the RSV F protein encoded by the adenoviral vector and the soluble RSV F protein comprise one or more stabilizing mutations as compared to a wild-type RSV F protein, in particular an RSV F protein comprising the amino acid sequence of SEQ ID NO: 1.

In a preferred embodiment, the RSV F protein encoded by the adenoviral vector has the amino acid sequence of SEQ ID NO: 5.

In addition, or alternatively, the nucleic acid encoding the RSV F protein encoded by the adenoviral vector comprises nucleotide sequence of SEQ ID NO: 4.

The RSV F protein of the second immunogen component comprises the ectodomain of the recombinant RSV F protein encoded by the adenoviral vector in order to obtain a soluble RSV F protein. Thus, the transmembrane and cytoplasmic domains have been removed, and optionally replaced by a heterologous trimerization domain, such as e.g. a foldon domain linked to the C-terminus of the F1 domain, either directly or through a linker.

In certain preferred embodiments, the RSV F protein of the second immunogenic component is a soluble protein comprising an amino acid sequence of SEQ ID NO: 7.

In addition, or alternatively, the RSV F protein of the second immunogenic component is a soluble protein encoded by a nucleotide sequence of SEQ ID NO: 8.

In a preferred embodiment, the effective amount of the first immunogenic component comprises about 1×10¹¹ viral particles of the adenoviral vector per dose.

In certain embodiments, the effective amount of the second immunogenic component comprises about 150 ug of the RSV F protein per dose.

The method of the present invention may further comprise administering to the subject (c) an effective amount of the first immunogenic component comprising about 1×10¹⁰ to about 1×10¹² viral particles of the adenoviral vector per dose, and (d) an effective amount of the second immunogenic component comprising about 30 ug to about 300 ug of the RSV F protein per dose, after the initial administration.

According to particular embodiments, the human subject is susceptible to RSV infection. In certain embodiments, a human subject that is susceptible to RSV infection includes, but is not limited to, an elderly human subject, for example a human subject ≥50 years old, preferably ≥60 years old, ≥65 years old; a young human subject, for example a human subject ≤5 years old, ≤1 year old; and/or a human subject that is hospitalized or a human subject that has been treated with an antiviral compound but has shown an inadequate antiviral response. In certain embodiments, a human subject that is susceptible to RSV infections includes a subject at risk, including but not limited to, a human subject with chronic heart disease, chronic lung disease, and/or immunodeficiency.

In certain preferred embodiments, the human subject is at least 60 years old.

In certain preferred embodiments, the human subject is at least 65 years old.

In certain embodiments, administration of the immunogenic combination results in the prevention of reverse transcriptase polymerase chain reaction (RT PCR)-confirmed RSV-mediated lower respiratory tract disease (LRTD). In certain embodiments, administration of the immunogenic combination results in the reduction of reverse transcriptase polymerase chain reaction (RT PCR)-confirmed RSV-mediated lower respiratory tract disease (LRTD), as compared to subjects which have not been administered the vaccine combination.

In addition, or alternatively, the protective immune response is characterized by an absent or reduced RSV viral load in the nasal track and/or lungs of the subject upon exposure to RSV.

In addition, or alternatively, the protective immune response is characterized by an absent or reduced RSV clinical symptom in the subject upon exposure to RSV.

In addition, or alternatively, the protective immune response is characterized by the presence of neutralizing antibodies to RSV and/or protective immunity against RSV.

In certain preferred embodiments, the method has an acceptable safety profile.

The application in particular relates to methods for safely preventing infection and/or replication of RSV in a human subject in need thereof, comprising prophylactically administering intramuscularly to the subject (a) an effective amount of a first immunogenic component, comprising about 1×10¹⁰ to about 1×10¹² viral particles per dose of an adenoviral vector comprising a nucleic acid encoding an RSV F protein having the amino acid sequence of SEQ ID NO: 5, wherein the adenoviral vector is replication-incompetent, and (b) an effective amount of a second immunogenic component, comprising about 30 ug to about 250 ug per dose of an RSV F protein having the amino acid sequence of SEQ ID NO: 7, and wherein (a) and (b) are co-administered.

The application also relates to methods of preventing or reducing reverse transcriptase polymerase chain reaction (RT PCR)-confirmed RSV-mediated lower respiratory tract disease (LRTD) in a human subject in need thereof, comprising prophylactically administering intramuscularly to the subject (a) an effective amount of a first immunogenic component, comprising about 1×1010 to about 1×1012 viral particles per dose of an adenoviral vector comprising a nucleic acid encoding an RSV F protein having the amino acid sequence of SEQ ID NO: 5, wherein the adenoviral vector is replication-incompetent, and (b) an effective amount of a second immunogenic component, comprising about 30 ug to about 250 ug per dose of an RSV F protein having the amino acid sequence of SEQ ID NO: 7, and wherein (a) and (b) are co-administered.

In these embodiments, the adenoviral vector may be a replication-incompetent Ad26 adenoviral vector having a deletion of the E1 region and the E3 region.

In certain preferred embodiments, the nucleic acid encoding the RSV F protein comprises the nucleotide sequence of SEQ ID NO: 4.

In certain embodiments, the effective amount of the first immunogenic component comprises about 1×10¹¹ viral particles of the adenoviral vector per dose.

In certain embodiments, the effective amount of the second immunogenic component comprises about 150 ug of the RSV F protein per dose.

In certain embodiments, the method further comprises administering to the subject (c) an effective amount of the first immunogenic component comprising about 1×10¹⁰ to about 1×10¹² viral particles of the adenoviral vector per dose, and (d) an effective amount of the second immunogenic component comprising about 30 ug to about 250 ug of the RSV F protein per dose, after the initial administration.

The invention furthermore provides a combination, such as e.g. a kit, comprising (a) a first immunogenic component, comprising an adenoviral vector comprising a nucleic acid encoding an RSV F protein that is stabilized in a pre-fusion conformation as described herein, wherein the effective amount of the first immunogenic component comprises about 1×10¹⁰ to about 1×10¹² viral particles of the adenoviral vector per dose, and (b) a second immunogenic component, comprising an RSV F protein that is stabilized in a pre-fusion conformation as described herein, wherein the effective amount of the second immunogenic component comprises about 30 ug to about 250 ug of the RSV F protein per dose. The combination can be used for inducing a protective immune response against RSV infection in a human subject in need thereof.

In another general aspect, the application describes products containing a combination of (a) a first immunogenic component comprising an adenoviral vector comprising a nucleic acid encoding an RSV F protein that is stabilized in a pre-fusion conformation as described herein, and (b) a second immunogenic component comprising an RSV F protein that is stabilized in a pre-fusion conformation as described herein, for simultaneous, separate or sequential use in inducing a protective immune response against RSV infection in a human subject in need thereof, preferably, the first and second immunogen components are co-administered, more preferably, the first immunogen component is administered at an effective amount of about 1×10¹⁰ to about 1×10¹² viral particles of the adenoviral vector per dose, and the second immunogenic component is administered at an effective amount of about 30 ug to about 300 ug of the RSV F protein per dose.

In preferred embodiments, the combination results in the prevention or reduction of reverse transcriptase polymerase chain reaction (RT PCR)-confirmed RSV-mediated lower respiratory tract disease (LRTD).

### BRIEF DESCRIPTION OF THE FIGURES

The foregoing summary, as well as the following detailed description of preferred embodiments of the present application, will be better understood when read in conjunction with the appended drawings. It should be understood, however, that the application is not limited to the precise embodiments shown in the drawings.

FIG. 1: Schematic representation of the RSV F protein precursor F0, RSV F mature processed and RSV preF protein. The two domains (F1 and F2), transmembrane domain (TM), foldon domain (FD), furin cleavage sites, N-glycan sites and interchain disulfide bonds of the proteins are shown. The 5 amino acid mutations in the RSV preF protein are also identified.

FIG. 2 shows plots of RSV A2 viral neutralizing antibody titers (VNT) at day 28 and at day 42 in naïve mice after a first and second immunization (day 0 and day 28, respectively) with RSV pre-F protein and/or Ad26.RSV.preF;

FIG. 3 shows pre-F and post-F binding antibody titers after prime-boost immunization with RSV pre-F protein and/or Ad26.RSV.preF in naive mice;

FIG. 4 shows cellular immune responses, as measured by IFNγ ELISPOT, after prime-boost immunization with RSV preF protein and/or Ad26.RSV.preF in naive mice;

FIG. 5 shows CD4+ T cell intracellular cytokine staining after prime-boost immunization with RSV preF protein and/or Ad26.RSV.preF in naive mice;

FIG. 6 shows CD8+ T cell intracellular cytokine staining after prime-boost immunization with RSV preF protein and/or Ad26.RSV.preF in naive mice;

FIG. 7 shows virus neutralization after prime-boost immunization with Ad26.RSV.preF or a combination of Ad26.RSV.preF with RSV preF protein in naive mice;

FIG. 8 shows pre-F and post-F binding antibody titers after prime-boost immunization with Ad26.RSV.preF or a combination of Ad26.RSV.preF with RSV preF protein in naive mice;

FIG. 9 shows cellular immune responses, as measured by IFNγ ELISPOT, after prime-boost immunization with Ad26.RSV.preF or a combination of Ad26.RSV.preF with RSV preF protein in naive mice;

FIG. 10 shows CD4+ T cell intracellular cytokine staining after prime-boost immunization with Ad26.RSV.preF or a combination of Ad26.RSV.preF with RSV preF protein in naive mice;

FIG. 11 shows CD8+ T cell intracellular cytokine staining after prime-boost immunization with Ad26.RSV.preF or a combination of Ad26.RSV.preF with RSV preF protein in naive mice;

FIG. 12 shows virus neutralization after single immunization with RSV preF protein and/or Ad26.RSV.preF in RSV pre-exposed mice;

FIG. 13 shows pre-F and post-F binding antibody titers after single immunization with RSV preF protein and/or Ad26.RSV.preF in RSV pre-exposed mice;

FIG. 14 shows cellular immune responses, as measured by IFNγ ELISPOT, after single immunization with RSV preF protein and/or Ad26.RSV.preF in RSV pre-exposed mice;

FIG. 15 shows CD4+ and CD8+ T cell intracellular cytokine staining after single immunization with RSV preF protein and/or Ad26.RSV.preF in RSV pre-exposed mice;

FIG. 16 shows virus neutralization after prime-boost immunization with RSV preF protein and/or Ad26.RSV.preF in pre-exposed mice;

FIG. 17 shows pre-F and post-F binding antibody titers after prime-boost immunization with RSV pre-F protein and/or Ad26.RSV.preF in pre-exposed mice;

FIG. 18 shows CD4+ and CD8+ T cell intracellular cytokine staining after prime-boost immunization with RSV preF protein and/or Ad26.RSV.preF in RSV pre-exposed mice FIG. 19 shows virus neutralization after single immunization with RSV preF protein and/or Ad26.RSV.preF in pre-exposed non-human primates (NHP);

FIG. 20 shows cellular immune responses after single immunization with RSV preF protein and/or Ad26.RSV.preF in pre-exposed NHP;

FIG. 21: Primary Efficacy Analysis: Percentage of participants with RT-PCR confirmed RSV-mediated LRTD according to each of the 3 Case Definitions and Vaccine Efficacy of their first occurrence; Per Protocol Efficacy set;

Case Definition 1: ≥3 symptoms of LRTI+RT-PCR confirmation for RSV

Case Definition 2: ≥2 symptoms of LRTI+RT-PCR confirmation for RSV

Case Definition 3: ≥2 symptoms of LRTI, OR ≥1 symptom of LRTI combined with ≥1 systemic symptom+RT-PCR confirmation for RSV

Vaccine efficacy is calculated based an exact Poisson regression with the event rate, defined as the number of cases over the follow-up time (offset) as dependent variable and the vaccination group and age and being at increased risk for severe RSV ARI (both as stratified) as independent variables. The confidence interval is adjusted to account for multiple endpoints. All subject data up to May 15, 2020 are included;

FIG. 22: Sensitivity analyses of the primary analysis—CD1 (≥3 symptoms of LRTI+RT-PCR confirmation of RSV);

FIG. 23: AUC of the total RiiQ Respiratory and Systemic Symptom Score, Case Definition Score and Impact of Daily Activity Score corresponding to RT-PCR confirmed RSV ARIs; Per Protocol Analysis Set;

FIG. 24: Kaplan-Meier of the number of days a participant took to return to its usual health; Per Protocol Efficacy set, Restricted to Participants with an RT-PCR Confirmed RSV ARI.

FIG. 25: Neutralizing Antibodies Against RSV A2 (A), pre-F ELISA Titers (B), and pre-F ELISpot Responses (C) Over Time Post Single Vaccination with Ad26.RSV.preF/RSV preF Protein (1×1011 vp/150 μg) (Green) and Placebo (Grey) (Selected Groups from Study VAC18193RSV1004, Cohort 2). ELISA=enzyme-linked immunosorbent assay; ELISpot=enzyme-linked immunospot; HD=high dose (1×1011 vp/150 μg); IgG=immunoglobulin G; IC50=50% inhibitory concentration; NAb=neutralizing antibodies; SFU/10\hat{ }6 PBMC=spot-forming units per million peripheral blood mononuclear cells; pre F=pre-fusion; vp=virus particles.

FIG. 26: Pre-F ELISA over Time With and Without Revaccination (Study VAC18193RSV1004, Cohort 3). Legend vaccine regimens:

Mix/Mix: Ad26.RSV.preF/RSV preF protein mix 1×1011 vp/150 μg on Day 1 and on Day 365. Mix/Pbo: Ad26.RSV.preF/RSV preF protein mix 1×1011 vp/150 μg on Day 1 and placebo on Day 365. CI=confidence interval; Nbas=number of participants at baseline; Pbo=placebo; pre-F ELISA=pre-fusion enzyme-linked immunosorbent assay; pre-F IgG=pre-fusion immunoglobulin G; vp=virus particles.

FIG. 27: VNA A2 over Time with and without Revaccination (Study VAC18193RSV1004, Cohort 3). Legend vaccine regimens:

Mix/Mix: Ad26.RSV.preF/RSV preF protein mix 1×1011 vp/150 μg on Day 1 and Day 365. Mix/Pbo: Ad26.RSV.preF/RSV preF protein mix 1×1011 vp/150 μg on Day 1 and placebo on Day 365. CI=confidence interval; IC50=50% inhibitory concentration; Nbas=number of participants at baseline; Pbo=placebo; VNA A2=virus neutralization assay for RSV A2; vp=virus particles.

FIG. 28: ELISpot over Time with and without Revaccination (Study VAC18193RSV1004, Cohort 3): Restricted to Participants with Day 393 Data. Legend vaccine regimens: Mix/Mix: Ad26.RSV.preF/RSV preF protein mix 1×1011 vp/150 μg on Day 1 and Day 365. Mix/Pbo: Ad26.RSV.preF/RSV preF protein mix 1×1011 vp/150 μg on Day 1 and placebo on Day 365. ELISpot=enzyme-linked immune absorbent spot; IFN=interferon; Nbas=number of participants at baseline; Q=quartile; SFU/10\hat{ }6 PBMC=spot-forming units per million peripheral blood mononuclear cells; vp=virus particles.

FIG. 29: Pre-F ELISA over Time with and without Revaccination (Study VAC18193RSV2001, revaccination cohort A).

FIG. 30: VNA_A2 over Time with and without Revaccination (Study VAC18193RSV2001, revaccination cohort A).

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

Although respiratory syncytial virus (RSV) infects people throughout life, most individuals fail to mount a long lasting protective immune response. In addition, in the elderly, the waning immune response contributes to increased susceptibility to severe disease after RSV infection, causing significant morbidity and mortality. There are indications in the literature that both neutralizing antibody and T-cell mediated protection play a role in preventing RSV infection. It is therefore believed that a successful RSV vaccine, in particular a successful vaccine for the elderly, should elicit both potent neutralizing antibody levels and induce a robust T-cell response.

Recently, stabilized pre-fusion RSV F proteins have been described with a unique set of amino acid mutations, as compared to the wild type RSV F protein from the RSV A2 strain (Genbank ACO83301.1) (see e.g. WO2014/174018, WO2017/174564 and WO2017/174568, the content of each of which is herein incorporated by reference in its entirety). By demonstrating specific binding to pre-fusion specific antibodies in vitro, it was shown that the RSV F protein antigen exists in the pre-fusion conformation and that the pre-fusion conformation was stable. Pre-clinical data showed that administration of the pre-fusion RSV F proteins induced virus neutralizing antibodies in both mice and cotton rats. Non-adjuvanted RSV preF protein induces very low T cell responses in mice. In cotton rats, prime boost immunization induced protection after intranasal challenge with the RSV A2 strain 3 weeks after boost immunization. Cotton rats immunized with pre-fusion RSV F proteins showed lower virus titer in the lung and nose 5 days after challenge compared with cotton rats immunized with post-fusion RSV F protein ((Krarup et al. Nat Comm 6, Article number: 8143, 2015).

In addition, human recombinant adenoviral vectors comprising DNA encoding for the RSV F protein in post-fusion confirmation induce virus neutralizing titers and T cell responses in mice after a single immunization. Prime immunization or heterologous prime boost immunization with adenoviral vector serotypes 26 and 35 encoding the post-fusion RSV F protein induced protection against intranasal challenge with RSV A2 or B15/97 in cotton rats (Widjojoatmodjo et al., Vaccine 33(41):5406-5414, 2015). Human recombinant adenoviral vectors comprising DNA encoding RSV F proteins in the pre-fusion conformation have been described in WO2014/174018 and WO2017/174564, the content of each of which is herein incorporated by reference in its entirety. In addition, it has been demonstrated that Ad26.RSV.preF had an acceptable safety profile and elicited sustained humoral and cellular immune responses after a single immunization in older adults (Williams et al., J Infect Dis 2020 Apr. 22; doi: 10.1093/infdis/jiaa193).

The present application describes compositions and methods with increased immunogenic efficacy. More specifically, the application describes efficacious immunogenic combinations for concurrent administration, that elicit both potent B and T cell responses, thereby enhancing immunogenicity, and ultimately protection, against respiratory syncytial virus (RSV) infection.

The present application thus provides methods for inducing a protective immune response against respiratory syncytial virus (RSV) infection in a human subject in need thereof, comprising administering to the subject (a) an effective amount of a first immunogenic component, comprising an adenoviral vector comprising a nucleic acid encoding an RSV F protein that is stabilized in a pre-fusion conformation, and (b) an effective amount of a second immunogenic component, comprising an RSV F protein that is stabilized in a pre-fusion conformation.

The immunogenic components are preferably administered concurrently, and the immunogenic combination elicits both potent B and T cell responses, thereby enhancing immunogenicity, safety, and ultimately protection against RSV.

In certain embodiments, the first and second immunogenic components are formulated in different compositions, which are mixed prior to co-administration. The first and second immunogenic components may however also be co-formulated in one composition.

In certain preferred embodiments, the immunogenic components are administered intramuscularly, i.e. by intramuscular injection As used herein, the term “RSV fusion protein,” “RSV F protein,” “RSV fusion protein” or “RSV F protein” refers to a fusion (F) protein of any group, subgroup, isolate, type, or strain of respiratory syncytial virus (RSV). RSV exists as a single serotype having two antigenic subgroups, A and B. Examples of RSV F protein include, but are not limited to, RSV F from RSV A, e.g. RSV A1 F protein and RSV A2 F protein, and RSV F from RSV B, e.g. RSV B1 F protein and RSV B2 F protein. As used herein, the term “RSV F protein” includes proteins comprising mutations, e.g., point mutations, fragments, insertions, deletions and splice variants of full-length wild type RSV F protein.

According to the invention, the recombinant RSV F protein encoded by the adenoviral vector and the soluble RSV F protein have been stabilized in the pre-fusion conformation. According to particular embodiments, the RSV F proteins that are stabilized in the pre-fusion conformation are derived from an RSV A strain. In certain embodiments the RSV F proteins are derived from the RSV A2 strain (Genbank ACO83301.1), RSV F proteins that have been stabilized in the pre-fusion conformation and that are useful in the application are RSV F proteins having at least one mutation as compared to a wild type RSV F protein, in particular as compared to the RSV F protein having the amino acid sequence of SEQ ID NO: 1. According to particular embodiments, RSV F proteins that are stabilized in the pre-fusion conformation that are useful according to the invention comprise at least one mutation selected from the group consisting of K66E, N671, 176V, S215P, and D486N. In a preferred embodiment, the RSV F proteins that are stabilized in the pre-fusion conformation according to the invention comprise the mutations K66E, N671, 176V, S215P, and D486N. It is again to be understood that for the numbering of the amino acid positions reference is made to SEQ ID NO: 1.

The RSV F proteins that are stabilized in the pre-fusion conformation comprise at least one epitope that is recognized by a pre-fusion specific monoclonal antibody, e.g. CR9501. CR9501 comprises the binding regions of the antibodies referred to as 58C5 in WO2011/020079 and WO2012/006596, which binds specifically to RSV F protein in its pre-fusion conformation and not to the post-fusion conformation.

In a preferred embodiment, the RSV F protein encoded by the adenoviral vector has the amino acid sequence of SEQ ID NO: 5.

In addition, or alternatively, the nucleic acid encoding the RSV F protein encoded by the adenoviral vector comprises nucleotide sequence of SEQ ID NO: 4. It is understood by a skilled person that numerous different nucleic acid molecules can encode the same protein as a result of the degeneracy of the genetic code. It is also understood that skilled persons can, using routine techniques, make nucleotide substitutions that do not affect the protein sequence encoded by the polynucleotides described there to reflect the codon usage of any particular host organism in which the proteins are to be expressed. Therefore, unless otherwise specified, a “nucleic acid molecule encoding an amino acid sequence” includes all nucleotide sequences that are degenerate versions of each other and that encode the same amino acid sequence. Nucleotide sequences that encode proteins and RNA can include introns. Sequences herein are provided from 5′ to 3′ direction, as custom in the art.

An adenovirus (or adenoviral vector) according to the invention belongs to the family of the Adenoviridae, and preferably is one that belongs to the genus Mastadenovirus. It can be a human adenovirus, but also an adenovirus that infects other species, including but not limited to a bovine adenovirus (e.g. bovine adenovirus 3, BAdV3), a canine adenovirus (e.g. CAdV2), a porcine adenovirus (e.g. PAdV3 or 5), or a simian adenovirus (which includes a monkey adenovirus and an ape adenovirus, such as a chimpanzee adenovirus or a gorilla adenovirus). Preferably, the adenovirus is a human adenovirus (HAdV, or AdHu), or a simian adenovirus such as chimpanzee or gorilla adenovirus (ChAd, AdCh, or SAdV), or a rhesus monkey adenovirus (RhAd). In the invention, a human adenovirus is meant if referred to as Ad without indication of species, e.g. the brief notation “Ad26” means the same as HAdV26, which is human adenovirus serotype 26. Also as used herein, the notation “rAd” means recombinant adenovirus, e.g., “rAd26” refers to recombinant human adenovirus 26.

Most advanced studies have been performed using human adenoviruses, and human adenoviruses are preferred according to certain aspects of the invention. In certain preferred embodiments, a recombinant adenovirus according to the invention is based upon a human adenovirus. In preferred embodiments, the recombinant adenovirus is based upon a human adenovirus serotype 5, 11, 26, 34, 35, 48, 49, 50, 52, etc. According to a particularly preferred embodiment of the invention, an adenovirus is a human adenovirus of serotype 26. Advantages of these serotypes include a low seroprevalence and/or low pre-existing neutralizing antibody titers in the human population, and experience with use in human subjects in clinical trials.

Simian adenoviruses generally also have a low seroprevalence and/or low pre-existing neutralizing antibody titers in the human population, and a significant amount of work has been reported using chimpanzee adenovirus vectors (e.g. U.S. Pat. No. 6,083,716; WO 2005/071093; WO 2010/086189; WO 2010085984; Farina et al, 2001, J Virol 75: 11603-13; Cohen et al, 2002, J Gen Virol 83: 151-55; Kobinger et al, 2006, Virology 346: 394-401; Tatsis et al., 2007, Molecular Therapy 15: 608-17; see also review by Bangari and Mittal, 2006, Vaccine 24: 849-62; and review by Lasaro and Ertl, 2009, Mol Ther 17: 1333-39). Hence, in other embodiments, the recombinant adenovirus according to the invention is based upon a simian adenovirus, e.g. a chimpanzee adenovirus. In certain embodiments, the recombinant adenovirus is based upon simian adenovirus type 1, 7, 8, 21, 22, 23, 24, 25, 26, 27.1, 28.1, 29, 30, 31.1, 32, 33, 34, 35.1, 36, 37.2, 39, 40.1, 41.1, 42.1, 43, 44, 45, 46, 48, 49, 50 or SA7P. In certain embodiments, the recombinant adenovirus is based upon a chimpanzee adenovirus such as ChAdOx 1 (see e.g. WO 2012/172277), or ChAdOx 2 (see e.g. WO 2018/215766). In certain embodiments, the recombinant adenovirus is based upon a chimpanzee adenovirus such as BZ28 (see e.g. WO 2019/086466). In certain embodiments, the recombinant adenovirus is based upon a gorilla adenovirus such as BLY6 (see e.g. WO 2019/086456), or BZ1 (see e.g. WO 2019/086466).

Preferably, the adenovirus vector is a replication deficient recombinant viral vector, such as rAd26, rAd35, rAd48, rAd5HVR48, etc.

In a preferred embodiment of the invention, the adenoviral vectors comprise capsid proteins from rare serotypes, e.g. including Ad26. In the typical embodiment, the vector is an rAd26 virus. An “adenovirus capsid protein” refers to a protein on the capsid of an adenovirus (e.g., Ad26, Ad35, rAd48, rAd5HVR48 vectors) that is involved in determining the serotype and/or tropism of a particular adenovirus. Adenoviral capsid proteins typically include the fiber, penton and/or hexon proteins. As used herein a “capsid protein” for a particular adenovirus, such as an “Ad26 capsid protein” can be, for example, a chimeric capsid protein that includes at least a part of an Ad26 capsid protein. In certain embodiments, the capsid protein is an entire capsid protein of Ad26. In certain embodiments, the hexon, penton and fiber are of Ad26.

One of ordinary skill in the art will recognize that elements derived from multiple serotypes can be combined in a single recombinant adenovirus vector. Thus, a chimeric adenovirus that combines desirable properties from different serotypes can be produced. Thus, in some embodiments, a chimeric adenovirus of the invention could combine the absence of pre-existing immunity of a first serotype with characteristics such as temperature stability, assembly, anchoring, production yield, redirected or improved infection, stability of the DNA in the target cell, and the like. See for example WO 2006/040330 for chimeric adenovirus Ad5HVR48, that includes an Ad5 backbone having partial capsids from Ad48, and also e.g. WO 2019/086461 for chimeric adenoviruses Ad26HVRPtr1, Ad26HVRPtr12, and Ad26HVRPtr13, that include an Ad26 virus backbone having partial capsid proteins of Ptr1, Ptr12, and Ptr13, respectively)

In certain embodiments the recombinant adenovirus vector useful in the invention is derived mainly or entirely from Ad26 (i.e., the vector is rAd26). In some embodiments, the adenovirus is replication deficient, e.g., because it contains a deletion in the E1 region of the genome. For adenoviruses being derived from non-group C adenovirus, such as Ad26 or Ad35, it is typical to exchange the E4-orf6 coding sequence of the adenovirus with the E4-orf6 of an adenovirus of human subgroup C such as Ad5. This allows propagation of such adenoviruses in well-known complementing cell lines that express the E1 genes of Ad5, such as for example 293 cells, PER.C6 cells, and the like (see, e.g. Havenga, et al., 2006, J Gen Virol 87: 2135-43; WO 03/104467). However, such adenoviruses will not be capable of replicating in non-complementing cells that do not express the E1 genes of Ad5.

The preparation of recombinant adenoviral vectors is well known in the art. Preparation of rAd26 vectors is described, for example, in WO 2007/104792 and in Abbink et al., (2007) Virol 81(9): 4654-63. Exemplary genome sequences of Ad26 are found in GenBank Accession EF 153474 and in SEQ ID NO: 1 of WO 2007/104792. Examples of vectors useful for the invention for instance include those described in WO2012/082918, the disclosure of which is incorporated herein by reference in its entirety.

Typically, a vector useful in the invention is produced using a nucleic acid comprising the entire recombinant adenoviral genome (e.g., a plasmid, cosmid, or baculovirus vector). Thus, the invention also provides isolated nucleic acid molecules that encode the adenoviral vectors of the invention. The nucleic acid molecules of the invention can be in the form of RNA or in the form of DNA obtained by cloning or produced synthetically. The DNA can be double-stranded or single-stranded.

The adenovirus vectors useful in the invention are typically replication deficient. In these embodiments, the virus is rendered replication deficient by deletion or inactivation of regions critical to replication of the virus, such as the E1 region. The regions can be substantially deleted or inactivated by, for example, inserting a gene of interest, such as a gene encoding an RSV F protein (usually linked to a promoter), within the region. In some embodiments, the vectors of the invention can contain deletions in other regions, such as the E2, E3 or E4 regions, or insertions of heterologous genes linked to a promoter within one or more of these regions. For E2- and/or E4-mutated adenoviruses, generally E2- and/or E4-complementing cell lines are used to generate recombinant adenoviruses. Mutations in the E3 region of the adenovirus need not be complemented by the cell line, since E3 is not required for replication.

A packaging cell line is typically used to produce sufficient amounts of adenovirus vectors for use in the invention. A packaging cell is a cell that comprises those genes that have been deleted or inactivated in a replication deficient vector, thus allowing the virus to replicate in the cell. Suitable packaging cell lines for adenoviruses with a deletion in the E1 region include, for example, PER.C6, 911, 293, and E1 A549.

According to the present invention, the vector is an adenovirus vector, and more preferably a rAd26 vector, most preferably a rAd26 vector with at least a deletion in the E1 region of the adenoviral genome, e.g. such as that described in Abbink, J Virol, 2007. 81(9): p. 4654-63, which is incorporated herein by reference. Typically, the nucleic acid sequence encoding the RSV F protein is cloned into the E1 and/or the E3 region of the adenoviral genome.

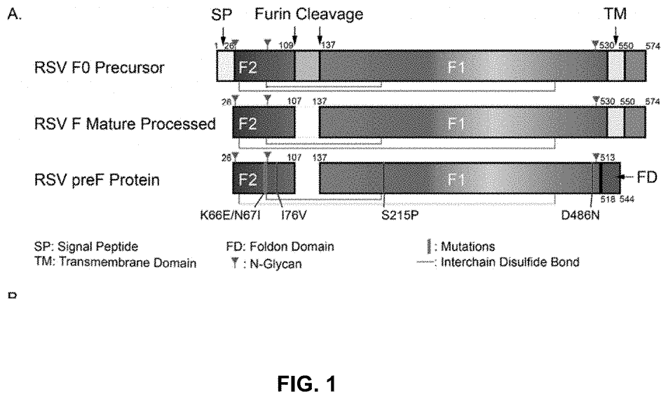

The RSV F protein of the second immunogen component typically comprises the ectodomain of the recombinant RSV F protein encoded by the adenoviral vector in order to obtain a soluble RSV F protein. RSV fusion (F) glycoprotein typically is synthesized as a F0 precursor which contains a signal peptide, F2 and F1 domains of the F protein and a peptide p27. The F0 is processed by furin or related host cellular proteases into F2 and F1 domains, the signal peptide and the p27 are removed. The F1 domain contains a transmembrane (TM) and cytoplasmic (CP) domains. F2 and F1 domains are connected by di-sulfide bridges. The F2-F1 heterodimers are organized on virions as trimeric spikes (FIG. 1). After processing, the processed mature RSV F protein encoded by the adenoviral vector comprises the F2 domain and F1 domains of SEQ ID NO: 4, which are linked by one or more disulfide bridges. The protein will not describe the signal peptide and the p27 peptide anymore.

The RSV preF protein of the second immunogenic component is a soluble recombinant construct of RSV F designed to be stable in the pre-fusion conformation. The RSV preF protein lacks the transmembrane and cytoplasmic domains. The T4 bacteriophage fibritin “foldon” (Fd) trimerization domain was added at the C-terminus to increase stability of the trimeric protein. Thus, the transmembrane and cytoplasmic domains have been removed, and optionally replaced by a heterologous trimerization domain, such as e.g. a foldon domain linked to the C-terminus of the the F1 domain, either directly or through a linker.

In certain embodiments, the trimerization domain comprises SEQ ID NO: 2 and is linked to amino acid residue 513 of the RSV F1 domain, either directly or through a linker. In certain embodiments, the linker comprises the amino acid sequence SAIG (SEQ ID NO: 3).

In certain preferred embodiments, the RSV F protein of the second immunogenic component is a soluble protein comprising an amino acid sequence of SEQ ID NO: 6 or 7.

In addition, or alternatively, the RSV F protein of the second immunogenic component is a soluble protein encoded by a nucleic acid having a nucleotide sequence of SEQID NO: 8.

In certain preferred embodiments, the first immunogenic component is or comprises a replication-incompetent adenovirus serotype 26 (Ad26) containing a deoxyribonucleic acid (DNA) transgene that encodes the pre-F conformation-stabilized membrane-bound F protein derived from the RSV A2 strain, preferably the pre-F protein of SEQ ID NO: 5, and the second immunogenic component is or comprises a recombinant, soluble, pre-F conformation-stabilized F protein derived from the RSV A2 strain, preferably the pre-F protein of SEQ ID NO: 6 or 7.

Immunogenic components described herein can be formulated as vaccines. As used herein, the term “vaccine” refers to a composition containing an active component effective to induce a certain degree of immunity in a subject against a certain pathogen or disease, which will result in at least a decrease, and up to complete absence, of the severity, duration or other manifestation of symptoms associated with infection by the pathogen or the disease. The vaccine(s) may induce an immune response against RSV, preferably both a humoral and cellular immune response against the F protein of RSV. According to embodiments, the vaccine(s) can be used to prevent serious lower respiratory tract disease leading to hospitalization and decrease the frequency of complications such as pneumonia, bronchitis and bronchiolitis due to RSV infection and replication in a subject. In certain embodiments, the vaccine(s) can be combination vaccine(s) that further comprises other components that induce a protective immune response, e.g. against other proteins of RSV and/or against other infectious agents, such as e.g. influenza. The administration of further active components can, for instance, be done by separate administration or by administering combination products of the vaccines of the application and the further active components.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. prevention or reduction of reverse transcriptase polymerase chain reaction (RT PCR)-Preferably, “protective immunity” or a “protective immune response” is shown by the prevention of PCR confirmed RSV-mediated lower respiratory tract disease (LRTD). Usually, a subject having a “protective immune response” or “protective immunity” against a certain agent will not die as a result of the infection with the agent.

As used herein, the term “induce” and variations thereof refers to any measurable increase in cellular activity. Induction of a protective immune response can include, for example, activation, proliferation, or maturation of a population of immune cells, increasing the production of a cytokine, and/or another indicator of increased immune function. In certain embodiments, induction of an immune response can include increasing the proliferation of B cells, producing antigen-specific antibodies, increasing the proliferation of antigen-specific T cells, improving dendritic cell antigen presentation and/or an increasing expression of certain cytokines, chemokines and co-stimulatory markers.

The ability to induce a protective immune response against RSV F protein can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by methods readily known in the art, e.g., by measurement of cytokine profiles secreted by activated effector cells including those derived from CD4+ and CD8+ T-cells (e.g. quantification of IL-4 or IFN gamma-producing cells by ELISPOT), by measuring PBMC proliferation, by measuring NK cell activity, by determination of the activation status of immune effector cells (e.g. T-cell proliferation assays by a classical [3H]thymidine uptake), by assaying for antigen-specific T lymphocytes in a sensitized subject (e.g. peptide-specific lysis in a cytotoxicity assay, etc.). Additionally, IgG and IgA antibody secreting cells with homing markers for local sites which can indicate trafficking to the gut, lung and nasal tissues can be measured in the blood at various times after immunization as an indication of local immunity, and IgG and IgA antibodies in nasal secretions can be measured; Fc function of antibodies and measurement of antibody interactions with cells such as PMNs, macrophages, and NK cells or with the complement system can be characterized; and single cell RNA sequencing analysis can be used to analyze B cell and T cell repertoires.

The ability to induce a protective immune response against RSV F protein can be determined by testing a biological sample (e.g., nasal wash, blood, plasma, serum, PBMCs, urine, saliva, feces, cerebral spinal fluid, bronchoalveolar lavage or lymph fluid) from the subject for the presence of antibodies, e.g. IgG or IgM antibodies, directed to the RSV F protein(s) administered in the composition, e.g. viral neutralizing antibody against RSV A2 (VNA A2), VNA RSV A Memphis 37b, RSV B, pre-F antibodies, post-F antibodies (see for example Harlow, 1989, Antibodies, Cold Spring Harbor Press). For example, titers of antibodies produced in response to administration of a composition providing an immunogen can be measured by enzyme-linked immunosorbent assay (ELISA), other ELISA-based assays (e.g., MSD-Meso Scale Discovery), dot blots, SDS-PAGE gels, ELISPOT, measurement of Fc interactions with complement, PMNs, macrophages and NK cells, with and without complement enhancement, or Antibody-Dependent Cellular Phagocytosis (ADCP) Assay. Exemplary methods are described in Example 1. According to particular embodiments, the induced immune response is characterized by neutralizing antibodies to RSV and/or protective immunity against RSV.

According to particular embodiments, the protective immune response is characterized by the presence of neutralizing antibodies to RSV and/or protective immunity against RSV, preferably detected 8 to 35 days after administration of the immunogenic components, such as 8, 9, 10, 11, 12, 13, 14, 15, 16, 17, 18, 19, 20, 21, 22, 23, 24, 25, 26, 27, 28, 29, 30, 31, 32, 33, 34 or 35 days after administration of the immunogenic components. More preferably, the neutralizing antibodies against RSV are detected about 6 months to 5 years after the administration of the immunogenic components, such as 6 months, 1 year, 2 years, 3 years, 4 years or 5 years after administration of the immunogenic components.

According to particular embodiments, the protective immune response is characterized by prevention of reverse transcriptase polymerase chain reaction (RT PCR)-confirmed RSV-mediated lower respiratory tract disease (LRTD). In certain embodiments, administration of the immunogenic combination results in the reduction of reverse transcriptase polymerase chain reaction (RT PCR)-confirmed RSV-mediated lower respiratory tract disease (LRTD), as compared to subjects which have not been administered the vaccine combination.

Exemplary methods are described in the Examples.

In addition, or alternatively, the protective immune response is characterized by an absent or reduced RSV clinical symptom in the subject upon exposure to RSV. RSV clinical symptoms include, for example, nasal congestion, sore throat, headache; cough, shortness of breath, wheezing, coughing up phlegm (sputum), fever or feeling feverish, body aches and pains, fatigue (tiredness), neck pain and loss of appetite.

As used herein, the term “acceptable safety profile” refers to a pattern of side effects that is within clinically acceptable limits as defined by regulatory authorities.

As used herein, the term “effective amount” refers to an amount of an active ingredient or component that elicits the desired biological or medicinal response in a subject. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the mode of administration, route of administration, target site, physiological state of the patient, other medications administered and the severity of disease. For example, the effective amount of immunogenic components also depends on whether adjuvant is also administered, with higher dosages being required in the absence of adjuvant.

According to embodiments, an effective amount of immunogenic component comprises an amount of immunogenic component that is sufficient to induce a protective immune response against RSV F protein with an acceptable safety profile. In particular embodiments, an effective amount of a first immunogenic component comprises from about 1×10¹⁰ to about 1×10¹² viral particles per dose, preferably about 1×10¹¹ viral particles per dose, of an adenoviral vector comprising a nucleic acid encoding an RSV F protein that is stabilized in a pre-fusion conformation. In particular embodiments, an effective amount of a second immunogenic component comprises from about 30 ug to about 300 ug per dose, preferably about 150 ug per dose, of an RSV F protein that is stabilized in a pre-fusion conformation.

According to embodiments, an effective amount of a first immunogenic component comprises about 1×10¹⁰ to about 1×10¹² viral particles per dose, such as about 1×10¹⁰ viral particles per dose, about 2×10¹⁰ viral particles per dose, about 3×10¹⁰ viral particles per dose, about 4×10¹⁰ viral particles per dose, about 5×10¹⁰ viral particles per dose, about 6×10¹⁰ viral particles per dose, about 7×10¹⁰ viral particles per dose, about 8×10¹⁰ viral particles per dose, about 9×10¹⁰ viral particles per dose, about 1×10¹¹ viral particles per dose, about 2×10¹¹ viral particles per dose, about 3×10¹¹ viral particles per dose, about 4×10¹¹ viral particles per dose, about 5×10¹¹ viral particles per dose, about 6×10¹¹ viral particles per dose, about 7×10¹¹ viral particles per dose, about 8×10¹¹ viral particles per dose, about 9×10¹¹ viral particles per dose, or about 1×10¹² viral particles per dose, of an adenoviral vector comprising a nucleic acid encoding an RSV F protein that is stabilized in a pre-fusion conformation.

In preferred embodiments, the effective amount of a first immunogenic component comprises about comprises between 5×10¹⁰ and 2×10¹¹ viral particles per dose, such as about 1×10¹¹ viral particles per dose, about 1.3×10¹¹ viral particles per dose or about 1.6×10¹¹ viral particles per dose.

Preferably the recombinant RSV F protein has an amino acid sequence of SEQ ID NO: 5 and the adenoviral vector is of serotype 26, such as a recombinant Ad26.

According to embodiments, an effective amount of a second immunogenic component comprises about 30 ug to about 300 ug per dose, such as about 30 ug per dose, about 40 ug per dose, about 50 ug per dose, about 60 ug per dose, about 70 ug per dose, about 80 ug per dose, about 90 ug per dose, about 100 ug per dose, about 110 ug per dose, about 120 ug per dose, about 130 ug per dose, about 140 ug per dose, about 150 ug per dose, about 160 ug per dose, about 170 ug per dose, about 180 ug per dose, about 190 ug per dose, about 200 ug per dose, about 225 ug per dose, or about 250 ug per dose, of an RSV F protein that is stabilized in a pre-fusion conformation. Preferably the recombinant RSV F protein has an amino acid sequence of SEQ ID NO: 6 or 7.

As used herein, the term “co-administered,” in the context of the administration of two or more immunogenic components or therapies to a subject, refers to the use of the two or more immunogenic components or therapies in combination and the two or more immunogenic components or therapies are administered to the subject within a period of 24 hours. In preferred embodiments, “co-administered” immunogenic components are pre-mixed and administered to a subject together at the same time. In other embodiments, “co-administered”immunogenic components are administered to a subject in separate compositions within 24 hours, such as within 12 hours, 10 hours, 8 hours, 6 hours, 4 hours, 2 hours, 1 hour or less.

In certain embodiments, the first and second immunogenic components are formulated, for example, with a pharmaceutically acceptable buffer, carrier, excipient and/or adjuvant, in different compositions. In other embodiments, the first and second immunogenic components are co-formulated, for example, with a pharmaceutically acceptable buffer, carrier, excipient and/or adjuvant, in a single composition for administration, for example admixed. Admixing can occur just prior to use, when the two components are manufactured and formulated, or any time between. In preferred embodiments, the first and second immunogenic components are co-formulated in a single composition for administration at the point of delivery shortly prior to administration, for example, bed side mixing, e.g. by using a multi-chamber syringe.

In certain embodiments, the first and second immunogenic components do not comprise an adjuvant.

According to particular embodiments, the human subject can be of any age, e.g. from about 1 month to 100 or more years old, e.g. from about 2 months to about 100 years old. When the immunogenic combination is administered to an infant, the composition can be administered one or more times. The first administration can be at or near the time of birth (e.g., on the day of or the day following birth), or within 1 week of birth or within about 2 weeks of birth. Alternatively, the first administration can be at about 4 weeks after birth, about 6 weeks after birth, about 2 months after birth, about 3 months after birth, about 4 months after birth, or later, such as about 6 months after birth, about 9 months after birth, or about 12 months after birth.

In certain embodiments, a human subject that is susceptible to RSV infection includes, but is not limited to, an elderly human subject, for example a human subject ≥50 years old, ≥60 years old, ≥65 years old; or a young human subject, for example a human subject ≤5 years old, ≤1 year old; and/or a human subject that is hospitalized or a human subject that has been treated with an antiviral compound but has shown an inadequate antiviral response. In certain embodiments, a human subject that is susceptible to RSV infections includes but is not limited to a human subject between 18 and 59 suffering from chronic heart disease, chronic lung disease, asthma and/or immunodeficiency.

In certain preferred embodiments, the human subject is at least 60 years old.

In certain preferred embodiments, the human subject is at least 65 years old.

According to particular embodiments, the first immunogenic component comprises a nucleic acid that encodes a protein antigen of RSV. Both deoxy-ribonucleic acids (DNA) and ribonucleic acids (RNA) are suitable. The nucleic acid can be included in a DNA or RNA vector, such as a replicable vector (e.g., a viral replicon, a self-amplifying nucleic acid), or in a virus (e.g., a live attenuated virus) or viral vector (e.g., replication proficient or replication deficient viral vector). Suitable viral vectors include but are not limited to an adenovirus, a modified vaccinia ankara virus (MVA), a paramyxovirus, a Newcastle disease virus, an alphavirus, a retrovirus, a lentivirus, an adeno-associated virus (AAV), a vesicular stomatitis virus, and a flavivirus. Optionally, the viral vector is replication defective. According to the application, the vector can be any vector that can be conveniently subjected to recombinant DNA procedures and can bring about expression of the nucleic acid molecule of the invention. The choice of the vector will typically depend on the compatibility of the vector with the host cell into which the vector is to be introduced.

According to particular embodiments, the first immunogenic component comprises an adenovirus comprising a nucleic acid molecule encoding an RSV F protein that is stabilized in the pre-fusion conformation.

In certain embodiments, the vector is a human recombinant adenovirus, also referred to as recombinant adenoviral vectors. The preparation of recombinant adenoviral vectors is well known in the art. The term “recombinant” for an adenovirus, as used herein implicates that it has been modified by the hand of man, e.g. it has altered terminal ends actively cloned therein and/or it comprises a heterologous gene, i.e. it is not a naturally occurring wild type adenovirus.

In certain embodiments, an adenoviral vector is deficient in at least one essential gene function of the E1 region, e.g. the Ela region and/or the E1b region, of the adenoviral genome that is required for viral replication. In certain embodiments, an adenoviral vector is deficient in at least part of the non-essential E3 region. In certain embodiments, the vector is deficient in at least one essential gene function of the E1 region and at least part of the non-essential E3 region. The adenoviral vector can be “multiply deficient,” meaning that the adenoviral vector is deficient in one or more essential gene functions in each of two or more regions of the adenoviral genome. For example, the aforementioned E1-deficient or E1-, E3-deficient adenoviral vectors can be further deficient in at least one essential gene of the E4 region and/or at least one essential gene of the E2 region (e.g., the E2A region and/or E2B region).

In certain embodiments, the recombinant adenovectors of the invention comprise as the 5′ terminal nucleotides the nucleotide sequence: CTATCTAT (SEQ ID NO: 9). These embodiments are advantageous because such vectors display improved replication in production processes, resulting in batches of adenovirus with improved homogeneity, as compared to vectors having the original 5′ terminal sequences (generally CATCATCA (SEQ ID NO: 10)) (see also patent application nos. PCT/EP2013/054846 and U.S. Ser. No. 13/794,318, entitled ‘Batches of recombinant adenovirus with altered terminal ends’ filed on 12 Mar. 2012 in the name of Crucell Holland B.V.), incorporated in its entirety by reference herein.

In certain embodiments, the nucleic acid molecule can encode a fragment of the pre-fusion F protein of RSV. The fragment can result from either or both of amino-terminal and carboxy-terminal deletions. The extent of deletion can be determined by a person skilled in the art to, for example, achieve better yield of the recombinant adenovirus. The fragment will be chosen to comprise an immunologically active fragment of the F protein, i.e. a part that will give rise to an immune response in a subject. This can be easily determined using in silico, in vitro and/or in vivo methods, all routine to the skilled person.

Recombinant adenovirus can be prepared and propagated in host cells, according to well-known methods, which entail cell culture of the host cells that are infected with the adenovirus. The cell culture can be any type of cell culture, including adherent cell culture, e.g. cells attached to the surface of a culture vessel or to microcarriers, as well as suspension culture.

According to particular embodiments, the second immunogenic component comprises an RSV F protein that is stabilized in the pre-fusion conformation. The pre-fusion RSV F proteins can be produced through recombinant DNA technology involving expression of the molecules in host cells, e.g., Chinese hamster ovary (CHO) cells, tumor cell lines, BHK cells, human cell lines such as HEK293 cells, PER.C6® cells, or yeast, fungi, insect cells, and the like, or transgenic animals or plants. In certain embodiments, the cells are from a multicellular organism; in certain embodiments, they are of vertebrate or invertebrate origin. In certain embodiments, the cells are mammalian cells. In certain embodiments, the cells are human cells. In general, the production of recombinant proteins in a host cell, such as the pre-fusion RSV F proteins of the disclosure, comprises the introduction of a heterologous nucleic acid molecule encoding the protein in expressible format into the host cell, culturing the cells under conditions conducive to expression of the nucleic acid molecule and allowing expression of the protein in the cell. The nucleic acid molecule encoding a protein in expressible format can be in the form of an expression cassette, and usually requires sequences capable of bringing about expression of the nucleic acid, such as enhancer(s), promoter, polyadenylation signal, and the like. The person skilled in the art is aware that various promoters can be used to obtain expression of a gene in host cells. Promoters can be constitutive or regulated, and can be obtained from various sources, including viruses, prokaryotic, or eukaryotic sources, or artificially designed.

Cell culture media are available from various vendors, and a suitable medium can be routinely chosen for a host cell to express the protein of interest, here, the pre-fusion RSV F proteins. The suitable medium may or may not contain serum.

A “heterologous nucleic acid molecule” (also referred to herein as “transgene”) is a nucleic acid molecule that is not naturally present in the host cell. It is introduced into, for instance, a vector by standard molecular biology techniques. A transgene is generally operably linked to expression control sequences. This can, for instance, be done by placing the nucleic acid encoding the transgene(s) under the control of a promoter. Further regulatory sequences can be added. Many promoters can be used for expression of a transgene(s), and are known to the skilled person, e.g., these can comprise viral, mammalian, synthetic promoters, and the like. A non-limiting example of a suitable promoter for obtaining expression in eukaryotic cells is a CMV-promoter (U.S. Pat. No. 5,385,839), e.g., the CMV immediate early promoter, for instance, comprising nt. −735 to +95 from the CMV immediate early gene enhancer/promoter. A polyadenylation signal, for example, the bovine growth hormone polyA signal (U.S. Pat. No. 5,122,458), can be present behind the transgene(s). Alternatively, several widely used expression vectors are available in the art and from commercial sources, e.g., the pcDNA and pEF vector series of INVITROGEN®, pMSCV and pTK-Hyg from BD Sciences, pCMV-Script from STRATAGENE™, etc., which can be used to recombinantly express the protein of interest, or to obtain suitable promoters and/or transcription terminator sequences, polyA sequences, and the like.

The cell culture can be any type of cell culture, including adherent cell culture, e.g., cells attached to the surface of a culture vessel or to microcarriers, as well as suspension culture. Most large-scale suspension cultures are operated as batch or fed-batch processes because they are the most straightforward to operate and scale up. Nowadays, continuous processes based on perfusion principles are becoming more common and are also suitable. Suitable culture media are also well known to the skilled person and can generally be obtained from commercial sources in large quantities, or custom-made according to standard protocols. Culturing can be done, for instance, in dishes, roller bottles or in bioreactors, using batch, fed-batch, continuous systems, and the like. Suitable conditions for culturing cells are known (see, e.g., Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9)).

In addition, or alternatively, the application provides methods for safely preventing infection and/or replication of RSV in a human subject in need thereof, comprising prophylactically administering intramuscularly to the subject (a) an effective amount of a first immunogenic component, comprising about 1×1010 to about 1×1012 viral particles per dose of an adenoviral vector comprising a nucleic acid encoding an RSV F protein having the amino acid sequence of SEQ ID NO: 5, wherein the adenoviral vector is replication-incompetent, and (b) an effective amount of a second immunogenic component, comprising about 30 ug to about 250 ug per dose of an RSV F protein having the amino acid sequence of SEQ ID NO: 7, and wherein (a) and (b) are co-administered.

The application also relates to methods of preventing or reducing reverse transcriptase polymerase chain reaction (RT PCR)-confirmed RSV-mediated lower respiratory tract disease (LRTD) in a human subject in need thereof, comprising prophylactically administering intramuscularly to the subject (a) an effective amount of a first immunogenic component, comprising about 1×10¹⁰ to about 1×10¹² viral particles per dose of an adenoviral vector comprising a nucleic acid encoding an RSV F protein having the amino acid sequence of SEQ ID NO: 5, wherein the adenoviral vector is replication-incompetent, and (b) an effective amount of a second immunogenic component, comprising about 30 ug to about 300 ug per dose of an RSV F protein having the amino acid sequence of SEQ ID NO: 7, and wherein (a) and (b) are co-administered.

In these embodiments, the adenoviral vector may be a replication-incompetent Ad26 adenoviral vector having a deletion of the E1 region and the E3 region.

In certain preferred embodiments, the nucleic acid encoding the RSV F protein comprises the nucleotide sequence of SEQ ID NO: 4.

In certain embodiments, the effective amount of the first immunogenic component comprises about 1×10¹¹ viral particles of the adenoviral vector per dose.

In certain embodiments, the effective amount of the second immunogenic component comprises about 150 ug of the RSV F protein per dose.

The methods described herein may further comprise administering to the subject (c) an effective amount of the first immunogenic component comprising about 1×10¹⁰ to about 1×10¹² viral particles of the adenoviral vector per dose, and (d) an effective amount of the second immunogenic component comprising about 30 ug to about 300 ug of the RSV F protein per dose, after the initial administration.

The interval between the administrations can vary. A typical regimen may comprise a first immunization with the combination as described herein followed by a second administration 1, 2, 4, 6, 8, 10 and 12 months later. Another regimen may entail one or 2 doses annually, prior to the RSV season.

It is readily appreciated by those skilled in the art that regimens for priming and boosting administrations can be adjusted based on the measured immune responses after the administrations. For example, boosting compositions are generally administered weeks or months after administration of the priming composition, for example, about 1 week, or 2-3 weeks or 4 weeks, or 8 weeks, or 16 weeks, or 20 weeks, or 24 weeks, or 28 weeks, or 32 weeks, or 36 weeks, or 40 weeks, or 44 weeks, or 48 weeks, or 52 weeks, or 56 weeks, or 60 weeks, or 64 weeks, or 68 weeks, or 72 weeks, or 76 weeks, or one to two, three, four of five years after administration of priming compositions.

According to particular embodiments, the first and/or second immunogenic components are formulated as pharmaceutical compositions. According to particular embodiments, the pharmaceutical compositions further comprise a pharmaceutically acceptable carrier or excipient. As used herein, the term “pharmaceutically acceptable” means that the carrier or excipient, at the dosages and concentrations employed, will not cause any unwanted or harmful effects in the subjects to which they are administered. Such pharmaceutically acceptable carriers and excipients are well known in the art (see Remington's Pharmaceutical Science (15th ed.), Mack Publishing Company, Easton, Pa., 1980). The preferred formulation of the pharmaceutical composition depends on the intended mode of administration and therapeutic application. The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or non-toxic, non-therapeutic, non-immunogenic stabilizers, and the like. It will be understood that the characteristics of the carrier, excipient or diluent will depend on the route of administration for a particular application.

In certain embodiments, pharmaceutical compositions according to the application further comprise one or more adjuvants. Adjuvants are known in the art to further increase the immune response to an applied antigenic determinant. The terms “adjuvant” and “immune stimulant” are used interchangeably herein and are defined as one or more substances that cause stimulation of the immune system. In this context, an adjuvant is used to enhance a protective immune response to the RSV F proteins of the pharmaceutical compositions. Examples of suitable adjuvants include aluminium salts such as aluminium hydroxide and/or aluminium phosphate; oil-emulsion compositions (or oil-in-water compositions), including squalene-water emulsions, such as MF59 (see e.g. WO 90/14837); saponin formulations, such as for example QS21 and Immunostimulating Complexes (ISCOMS) (see e.g. U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g. antibodies or fragments thereof (e.g. directed against the antigen itself or CDla, CD3, CD7, CD80) and ligands to receptors (e.g. CD40L, GMCSF, GCSF, etc.), which stimulate immune response upon interaction with recipient cells. It is also possible to use vector-encoded adjuvant, e.g. by using heterologous nucleic acid that encodes a fusion of the oligomerization domain of C4-binding protein (C4 bp) to the antigen of interest (e.g. Solabomi et al, 2008, Infect Immun 76: 3817-23). In certain embodiments, the first immunogenic component is formulated with an adjuvant. In other embodiments, the second immunogenic component is formulated with an adjuvant. In certain embodiments, both immunogenic components contain an adjuvant. Typically, the adjuvant is admixed (e.g., prior to administration or stably formulated) with the antigenic component. When the immunogenic combination is to be administered to a subject of a particular age group, the adjuvant is selected to be safe and effective in the subject or population of subjects. Thus, when formulating a immunogenic combination for administration to an elderly subject (such as a subject greater than 65 years of age), the adjuvant is selected to be safe and effective in elderly subjects. Similarly, when the combination immunogenic composition is intended for administration to neonatal or infant subjects (such as subjects between birth and the age of two years), the adjuvant is selected to be safe and effective in neonates and infants. In certain embodiments the pharmaceutical compositions comprise aluminium as an adjuvant, e.g. in the form of aluminium hydroxide, aluminium phosphate, aluminium potassium phosphate, or combinations thereof, in concentrations of 0.05-5 mg, e.g. 0.075-1.0 mg, of aluminium content per dose.

The pharmaceutical compositions can be used e.g. in stand-alone prophylaxis of a disease or condition caused by RSV, or in combination with other prophylactic and/or therapeutic treatments, such as (existing or future) vaccines, antiviral agents and/or monoclonal antibodies.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a pharmaceutical composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

Pharmaceutical compositions of the present application can be formulated according to methods known in the art in view of the present disclosure.

The application also provides methods for preventing infection and/or replication of RSV with an acceptable safety profile in a human subject in need thereof. In particular embodiments, the method comprises prophylactically administering to the subject (a) an effective amount of a first immunogenic component, comprising an adenoviral vector comprising a nucleic acid encoding an RSV F protein that is stabilized in a pre-fusion conformation, and (b) an effective amount of a second immunogenic component, comprising an RSV F protein that is stabilized in a pre-fusion conformation. This will reduce adverse effects resulting from RSV infection in a subject, and thus contribute to protection of the subject against such adverse effects upon administration of the pharmaceutical composition.

According to particular embodiments, the prevented infection and/or replication of RSV is characterized by absent or reduced RSV viral load in the nasal track and/or lungs of the subject, and/or by absent or reduced clinical symptoms of RSV infection upon exposure to RSV, as compared to that in a subject to whom the pharmaceutical composition was not administered, upon exposure to RSV. In certain embodiments, absent RSV viral load or absent adverse effects of RSV infection means reduced to such low levels that they are not clinically relevant.

According to particular embodiments, the prevented infection and/or replication of RSV is characterized by prevention or reduction of reverse transcriptase polymerase chain reaction (RT PCR)-confirmed RSV-mediated lower respiratory tract disease (LRTD) in the subject upon exposure to RSV.

In addition, or alternatively, the prevented infection and/or replication of RSV is characterized by the presence of neutralizing antibodies to RSV and/or protective immunity against RSV, preferably detected 8 to 35 days after administration of the pharmaceutical composition, such as 14, 15, 16, 17, 18, 19, 20, 21, 22, 23, 24, 25, 26, 27, 28, 29, 30, 31, 32, 33, 34 or 35 days after administration of the pharmaceutical composition. More preferably, the neutralizing antibodies against RSV are detected about 6 months to 5 years after the administration of the pharmaceutical composition, such as 6 months, 1 year, 2 years, 3 years, 4 years or 5 years after administration of the pharmaceutical composition.

In addition, or alternatively, the prevented infection and/or replication of RSV is characterized by a decrease in symptomatic disease as compared to that in a subject to whom the pharmaceutical composition was not administered, upon exposure to RSV.

In addition, or alternatively, the prevented infection and/or replication of RSV is characterized by a quicker return to health as compared to that in a subject to whom the pharmaceutical composition was not administered, upon exposure to RSV.

According to embodiments, an effective amount of pharmaceutical composition comprises an amount of pharmaceutical composition that is sufficient to prevent infection and/or replication of RSV with an acceptable safety profile. In particular embodiments, an effective amount of a first immunogenic component comprises from about 1×10¹⁰ to about 1×10¹² viral particles per dose, preferably about 1×10¹¹ viral particles per dose, of an adenoviral vector comprising a nucleic acid encoding an RSV F protein that is stabilized in a pre-fusion conformation. In particular embodiments, an effective amount of a second immunogenic component comprises from about 30 ug to about 300 ug per dose, preferably about 150 ug per dose, of an RSV F protein that is stabilized in a pre-fusion conformation.

According to embodiments, an effective amount of a first immunogenic component comprises about 1×10¹⁰ to about 1×10¹² viral particles per dose, such as about 1×10¹⁰ viral particles per dose, about 2×10¹⁰ viral particles per dose, about 3×10¹⁰ viral particles per dose, about 4×10¹⁰ viral particles per dose, about 5×10¹⁰ viral particles per dose, about 6×10¹⁰ viral particles per dose, about 7×10¹⁰ viral particles per dose, about 8×10¹⁰ viral particles per dose, about 9×10¹⁰ viral particles per dose, about 1×10¹¹ viral particles per dose, about 2×10¹¹ viral particles per dose, about 3×10¹¹ viral particles per dose, about 4×10¹¹ viral particles per dose, about 5×10¹¹ viral particles per dose, about 6×10¹¹ viral particles per dose, about 7×10¹¹ viral particles per dose, about 8×10¹¹ viral particles per dose, about 9×10¹¹ viral particles per dose, or about 1×10¹² viral particles per dose, of an adenoviral vector comprising a nucleic acid encoding an RSV F protein that is stabilized in a pre-fusion conformation.

In preferred embodiments, the effective amount of a first immunogenic component comprises about comprises between 5×10¹⁰ and 2×10¹¹ viral particles per dose, such as about 1×10¹¹ viral particles per dose, about 1.3×10¹¹ viral particles per dose or about 1.6×10¹¹ viral particles per dose.

Preferably the recombinant RSV F protein has an amino acid sequence of SEQ ID NO: 5, and the adenoviral vector is of serotype 26, such as a recombinant Ad26.

According to embodiments, an effective amount of a second immunogenic component comprises about 30 ug to about 300 ug per dose, such as about 30 ug per dose, about 40 ug per dose, about 50 ug per dose, about 60 ug per dose, about 70 ug per dose, about 80 ug per dose, about 90 ug per dose, about 100 ug per dose, about 110 ug per dose, about 120 ug per dose, about 130 ug per dose, about 140 ug per dose, about 150 ug per dose, about 160 ug per dose, about 170 ug per dose, about 180 ug per dose, about 190 ug per dose, about 200 ug per dose, about 225 ug per dose, or about 250 ug per dose, of a soluble RSV F protein that is stabilized in a pre-fusion conformation. Preferably the soluble recombinant RSV F protein has an amino acid sequence of SEQ ID NO: 6 or SEQ ID NO: 7. In addition, or alternatively, the soluble recombinant RSV F protein is encoded by a nucleic acid having a nucleotide sequence of SEQ ID NO: 8.

The application also provides methods for vaccinating a subject against RSV infection with an acceptable safety profile in a human subject in need thereof. In particular embodiments, the method comprises administering to the subject (a) an effective amount of a first immunogenic component, comprising an adenoviral vector comprising a nucleic acid encoding an RSV F protein that is stabilized in a pre-fusion conformation, and (b) an effective amount of a second immunogenic component, comprising an RSV F protein that is stabilized in a pre-fusion conformation.

According to embodiments, an effective amount of pharmaceutical composition comprises an amount of pharmaceutical composition that is sufficient to vaccinate a subject against RSV infection with an acceptable safety profile. In particular embodiments, an effective amount of a first immunogenic component comprises from about 1×10¹⁰ to about 1×10¹² viral particles per dose, preferably about 1×10¹¹ viral particles per dose, of an adenoviral vector comprising a nucleic acid encoding an RSV F protein that is stabilized in a pre-fusion conformation. In particular embodiments, an effective amount of a second immunogenic component comprises from about 30 ug to about 300 ug per dose, preferably about 150 ug per dose, of an RSV F protein that is stabilized in a pre-fusion conformation.

According to embodiments, an effective amount of a first immunogenic component comprises about 1×10¹⁰ to about 1×10¹² viral particles per dose, such as about 1×10¹⁰ viral particles per dose, about 2×10¹⁰ viral particles per dose, about 3×10¹⁰ viral particles per dose, about 4×10¹⁰ viral particles per dose, about 5×10¹⁰ viral particles per dose, about 6×10¹⁰ viral particles per dose, about 7×10¹⁰ viral particles per dose, about 8×10¹⁰ viral particles per dose, about 9×10¹⁰ viral particles per dose, about 1×10¹¹ viral particles per dose, about 2×10¹¹ viral particles per dose, about 3×10¹¹ viral particles per dose, about 4×10¹¹ viral particles per dose, about 5×10¹¹ viral particles per dose, about 6×10¹¹ viral particles per dose, about 7×10¹¹ viral particles per dose, about 8×10¹¹ viral particles per dose, about 9×10¹¹ viral particles per dose, or about 1×10¹² viral particles per dose, of an adenoviral vector comprising a nucleic acid encoding an RSV F protein that is stabilized in a pre-fusion conformation. Preferably the recombinant RSV F protein has an amino acid sequence of SEQ ID NO: 5, and the adenoviral vector is of serotype 26, such as a recombinant Ad26.

According to embodiments, an effective amount of a second immunogenic component comprises about 30 ug to about 300 ug per dose, such as about 30 ug per dose, about 40 ug per dose, about 50 ug per dose, about 60 ug per dose, about 70 ug per dose, about 80 ug per dose, about 90 ug per dose, about 100 ug per dose, about 110 ug per dose, about 120 ug per dose, about 130 ug per dose, about 140 ug per dose, about 150 ug per dose, about 160 ug per dose, about 170 ug per dose, about 180 ug per dose, about 190 ug per dose, about 200 ug per dose, about 225 ug per dose, or about 250 ug per dose, of a soluble RSV F protein that is stabilized in a pre-fusion conformation. Preferably the soluble recombinant RSV F protein has an amino acid sequence of SEQ ID NO: 6 or SEQ ID NO: 7. In addition, or alternatively, the soluble recombinant RSV F protein is encoded by a nucleic acid having a nucleotide sequence of SEQ ID NO: 8.

The application also provides immunogenic combinations (e.g. kits), or vaccine combinations, comprising (a) a first immunogenic component, comprising an adenoviral vector comprising a nucleic acid encoding an RSV F protein that is stabilized in a pre-fusion conformation as described herein, wherein the effective amount of the first immunogenic component comprises about 1×10¹⁰ to about 1×10¹² viral particles of the adenoviral vector per dose, and (b) a second immunogenic component, comprising an RSV F protein that is stabilized in a pre-fusion conformation as described herein, wherein the effective amount of the second immunogenic component comprises about 30 ug to about 300 ug of the RSV F protein per dose. The combination can be used for inducing a protective immune response against RSV infection in a human subject in need thereof. Preferably, the combination is used for the prevention of reverse transcriptase polymerase chain reaction (RT PCR)-confirmed RSV-mediated lower respiratory tract disease (LRTD).

The immunogenic components of the combinations can comprise co-formulated compositions or different compositions that separately provide each component. In certain embodiments, the combinations comprise the first immunogenic component and the second immunogenic component in one container. In other embodiments, combinations comprise the first immunogenic component and the second immunogenic component in separate containers. The container(s) can be, for example, one or more pre-filled syringe. Such a syringe can be a multi-chamber (e.g., dual-chamber) syringe. In certain embodiments, in the case of a multi-chamber syringe, the first immunogenic component is contained within one chamber, and the second immunogenic component is contained within a second chamber. Prior to administration, the two components can be admixed and then administered to the subject at the same site (e.g., through a single needle).

### EXAMPLES

The following examples of the application are intended to further illustrate the nature of the application. It should be understood that the following examples do not limit the invention and that the scope of the invention is to be determined by the appended claims.

### Example 1: Immunogenicity of Non-Adjuvanted RSV Pre-F Protein and Ad26.RSV.Pre-F in Naive Mice

In naive mice, the humoral and cellular immunogenicity of 5 μg or 0.5 μg non-adjuvanted RSV pre-F protein was measured when given together with a suboptimal dose of 1×10⁸ viral particles (vp) Ad26.RSV.pre-F in a homologous prime-boost schedule. In naïve mice, the (suboptimal) dose of 1×10⁸ vp Ad26.RSV.pre-F induced very low to undetectable virus neutralization titers (VNT) to the RSV A2 strain. The mixture contained the Ad26.RSV.pre-F buffer and the RSV pre-F protein buffer (PBS) at a ratio of 1:1. Comparison groups received PBS only or prime-boost immunizations with either RSV pre-F protein, or Ad26.RSV.pre-F.

Balb/c mice were primed and boost immunized intramuscularly (IM) with a mixture of 10⁸ vp Ad26.RSV.pre-F and 5 ug or 0.5 ug RSV pre-F protein (n=12 per group), or with 10⁸ vp Ad26.RSV.pre-F (n=12), or with 5 ug or 0.5 ug RSV pre-F protein (n=8 per group), or with PBS (n=8). The prime-boost interval was 4 weeks.

Neutralizing Antibody Responses

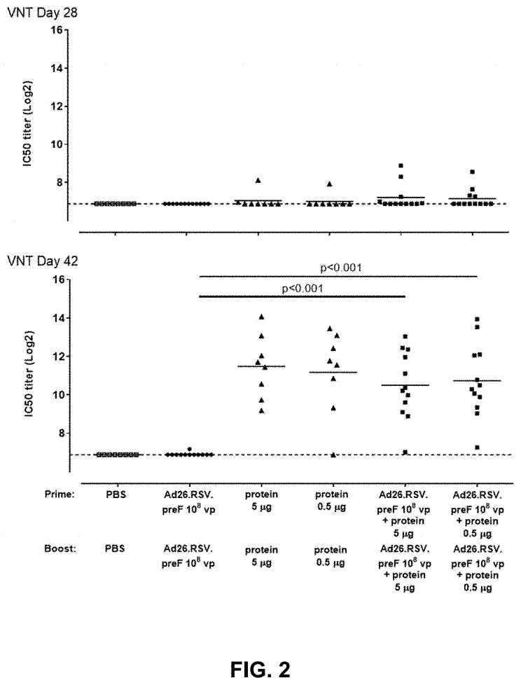

At 2 weeks post-boost immunization, animals were sacrificed, and sera was isolated. RSV A2 virus neutralizing titers were determined using a firefly luciferase reporter-based assay. The IC50 titers were calculated, and the results are shown in FIG. 2. The mean response per group is indicated with a horizontal line. The dashed line shows the lower limit of quantification of 6.88 log 2. Statistical analysis was performed with analysis of variance (ANOVA). In all groups, VNT were low to undetectable 4 weeks (Day 28) post prime immunization (FIG. 2, upper panel). Two weeks post-boost (Day 42), immunization with 5 ug and 0.5 ug doses of RSV preF protein alone induced VNT that were comparable between the 2 doses (FIG. 2, bottom panel).

In naive mice, the mixture of RSV pre-F protein and Ad26.RSV.pre-F induced higher VNT than a low (suboptimal) dose of Ad26.RSV.preF alone (1×10⁸ vp) at the 5 ug and 0.5 ug RSV pre-F protein doses tested (p<0.001, ANOVA). The VNT induced by RSV pr-eF protein alone was not significantly different compared with the VNT of the mixture of RSV pre-F protein and Ad26.RSV.preF (p=0.255, ANOVA across-dose comparison).

RSV Pre-F and Post-F Binding Antibody Responses

IgG antibodies to RSV pre-F and RSV post-F were measured by ELISA. Plates were coated with anti-RSV F followed by addition of RSV pre-F or RSV post-F protein. The plates were incubated with serially diluted samples followed by detection with anti-mouse IgG, and the optical density was measured.

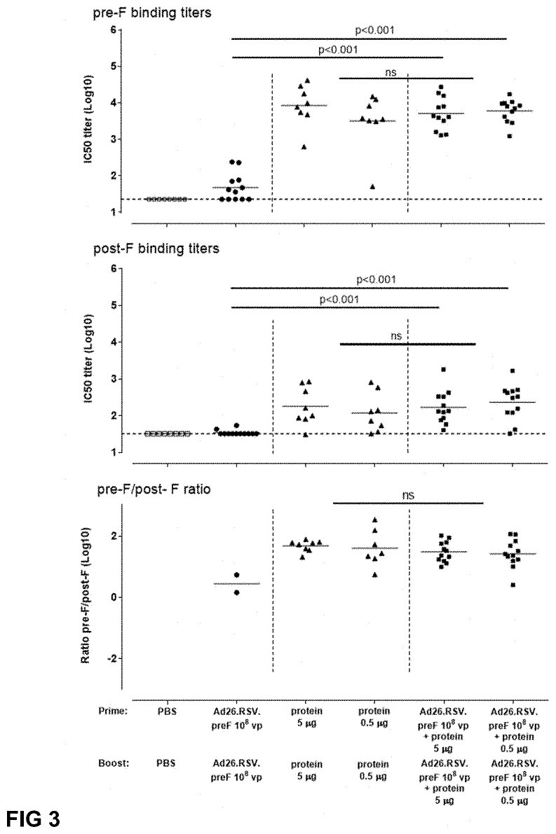

In all groups, RSV pre-F and post-F antibody titers were low to undetectable 4 weeks post prime immunization (data not shown). Two weeks post boost immunization, high RSV pre-F antibody titers were induced after immunization with 0.5 ug or 5 ug RSV pre-F protein. A mixture of RSV pre-F protein and Ad26.RSV.pre-F induced similar anti RSV pre-F titers to RSV pre-F protein alone (p=0.869, ANOVA across-dose comparison) (FIG. 3, top panel). Mice immunized with low dose Ad26.RSV.pre-F alone had low or undetectable RSV pre-F antibody titers, and the mixture of RSV pre-F protein and Ad26.RSV.pre-F induced significantly higher RSV pre-F antibody titers compared with Ad26.RSV.pre-F alone (p<0.001, ANOVA). A similar pattern of antibody induction was observed for post-F binding antibodies, although with lower titers than against RSV pre-F (FIG. 3, middle panel). Titers are given as the log 10 value of the IC50. The lower limit of quantification (LLoQ) is indicated with a dashed line. FIG. 3, bottom panel displays the ratio between preF and postF antibodies for all samples that showed preF and postF titers above LLoQ. The mean response per group is indicated with a horizontal line. Statistical comparison of Ad26.RSV.preF alone with the mixture was performed with analysis of variance (ANOVA) and the comparison of protein with mixture was compared with ANOVA across-dose comparison (ns=not significant).

Mice immunized with a mixture of RSV pre-F protein and Ad26.RSV.pre-F did not show a significantly different RSV pre-F/post-F binding antibody ratio compared with mice immunized with RSV preF protein alone (p=0.146, ANOVA across-dose comparison). A comparison with the Ad26.RSV.pre-F only group could not be made due to the undetectable titers in many animals from this group.

Cellular Responses

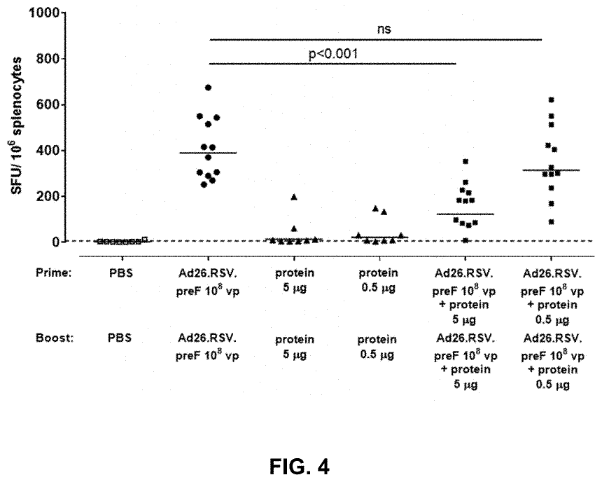

Cellular responses were measured in splenocytes taken 2 weeks post boost immunization. Splenocytes were isolated and stimulated with a peptide pool covering the RSV A2 F protein. The number of IFNγ spot forming units (SFU) per 10⁶ splenocytes was determined by enzyme-linked immunospot (ELISPOT) (FIG. 4). The geometric mean response per group is indicated with a horizontal line. The dashed line shows the limit of detection, defined as the 95% percentile of the SFU observed in non-stimulated splenocytes.

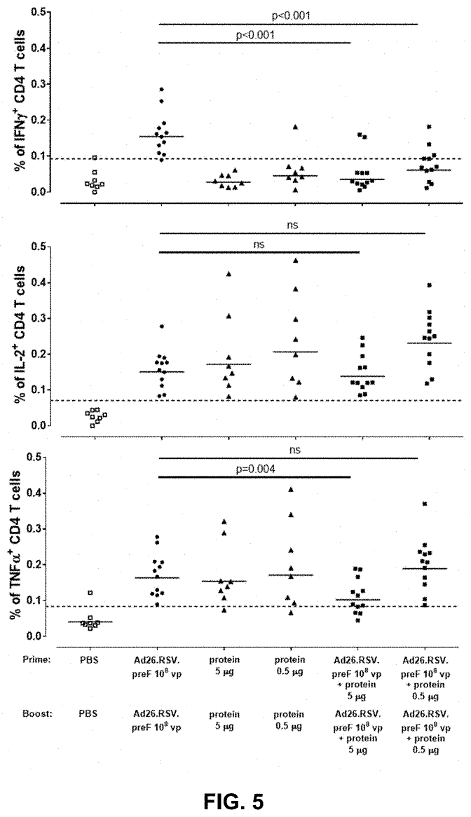

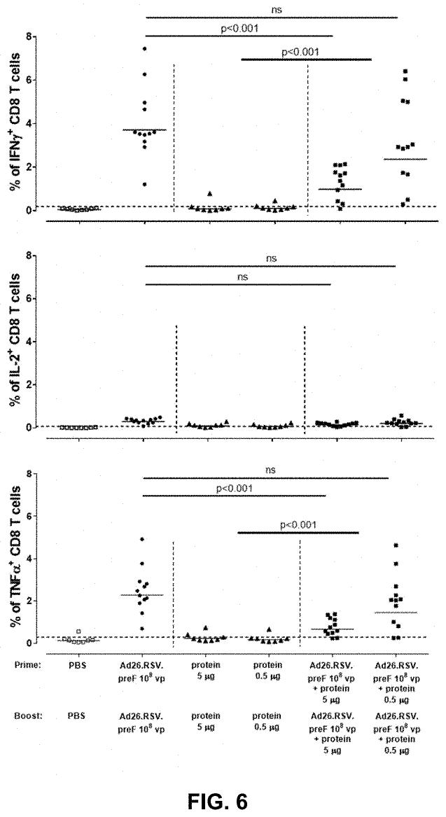

Prime and boost immunization with Ad26.RSV.pre-F only or when mixed with a low dose (0.5 ug) of RSV preF protein induced comparable ELISPOT IFNγ+ T cell responses (FIG. 3). A mixture of Ad26.RSV.preF with a higher dose (5 ug) of RSV preF protein gave a significantly lower IFNγ+ T cell response compared with Ad26.RSV.preF alone (p<0.001, ANOVA). Prime-boost immunization with RSV preF protein alone induced a negligible cellular response to RSV F. Similar results were seen in the ICS assay (FIG. 5 and FIG. 6). Immunization with Ad26.RSV.preF induced CD4+ and CD8+ T cells producing IFNγ, TNFα and IL-2. Immunization with a mixture of RSV preF protein and Ad26.RSV.preF resulted in reduced CD4+ and CD8+ T cell responses, in particular for the higher protein dose and the cell populations producing IFNγ and TNFα.

In FIG. 5, the percentage of cytokine positive CD3+CD4+ splenocytes measured by ICS is shown. The limit of detection (LOD) was defined as the mean background staining +3 standard deviations of medium controls. LOD CD3+CD4+ for IFNγ, TNFα and IL-2 were 0.09, 0.08 and 0.07, respectively. Statistical analysis was performed with analysis of variance (ANOVA) (ns=not significant).

In FIG. 6, the percentage of cytokine positive CD3+CD8+ splenocytes measured by ICS is shown. The limit of detection (LOD) was defined as the mean background staining+3 standard deviations of medium controls. LOD CD3+CD8+ for IFNγ, TNFα and IL-2 were 0.19, 0.29 and 0.07, respectively. Statistical analysis was performed with analysis of variance (ANOVA) or ANOVA with across-dose comparison (ns=not significant).

### Example 2: Immunogenicity of Various Ad26.RSV.Pre-F and RSV Pre-F Protein Mix Combinations in Mice

In naive mice, the humoral and cellular immunogenicity of a mixture of 1×10⁸ vp Ad26.RSV.pre-F and various RSV pre-F protein concentrations (15, 1.5, 0.15, and 0.015 ug) was compared with 1×10⁸ vp Ad26.RSV.pre-F alone following a homologous prime boost schedule in mice. Balb/c mice were prime- and boost-immunized IM with a mixture of 10⁸ viral particles (vp) Ad26.RSV.pre-F with 15, 1.5, 0.15, or 0.015 ug RSV preF protein, a mixture of 10⁹ vp Ad26.RSV.pre-F with 15 ug RSV pre-F protein, or with 10⁸ vp or 109Ad26.RSV.pre-F (n=6 per group), or with PBS (n=3). The mixture contained the Ad26.RSV.pre-F buffer and the RSV pre-F protein formulation buffer at a ratio of 1:1. Negative control group received a mix of the two formulation buffers at a ratio of 1:1. The prime-boost interval was 4 weeks. At 2 weeks post-boost immunization, animals were sacrificed, and sera were isolated.

Neutralizing Antibody Responses

RSV CL57 virus neutralizing titers were determined using a firefly luciferase reporter-based assay. The IC90 titers were calculated and the mean response per group is indicated with a horizontal line (FIG. 6). The dashed line shows the lower limit of quantification of 6.88 log 2. Statistical analysis was performed with analysis of variance (ANOVA).

Two weeks post-boost (Day 42), immunization with a mixture of 1×10⁸ vp Ad26.RSV.pre-F and 15, 1.5, 0.15, or 0.015 ug RSV preF protein induced significantly higher VNT compared with Ad26.RSV.preF alone (p≤0.018 ANOVA, sequential testing starting with the highest dose). A mix of 1×10⁹ vp Ad26.RSV.pre-F and 15 ug RSV pre-F protein showed higher VNT compared with 1×10⁹ vp Ad26.RSV.preF alone.

RSV Pre-F and Post-F Binding Antibody Responses

IgG antibodies to RSV pre-F and RSV post-F were measured by ELISA. Plates were coated with anti-RSV F followed by addition of RSV pre-F or RSV post-F protein. The plates were incubated with serially diluted samples followed by detection with anti-mouse IgG, and the optical density was measured.

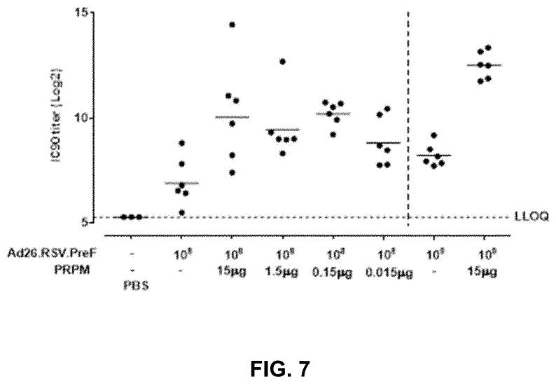

Titers are given as the log 10 value of the IC50 (FIG. 7). The lower limit of quantification (LLoQ) is indicated with a dashed line. The lower graph displays the ratio between preF and postF antibodies for all samples that showed preF and postF titers above LLoQ. The mean response per group is indicated with a horizontal line. Statistical comparison of Ad26.RSV.preF alone with the mixture was performed with analysis of variance (ANOVA) with sequential testing starting with the highest protein dose; ns=not significant.

Two weeks post boost immunization, mice receiving a suboptimal dose of Ad26.RSV.pre-F (10⁸ vp) showed low RSV pre-F antibody titers. Immunization with a mixture of Ad26.RSV.pre-F and RSV pre-F protein induced significantly higher RSV pre-F titer compared with Ad26.RSV.pre-F alone, for all RSV pre-F protein doses tested (p<0.001 for all, ANOVA). The mixture did not induce significantly higher RSV post-F titers compared with Ad26.RSV.pre-F alone. A significantly higher pre-F/post-F ratio compared with Ad26.RSV.pre-F alone was observed (p<0.001 for all, ANOVA). Similar findings were observed with a mixture of 10⁹ vp Ad26.RSV.pre-F and 15 ug RSV pre-F protein.

Cellular Responses

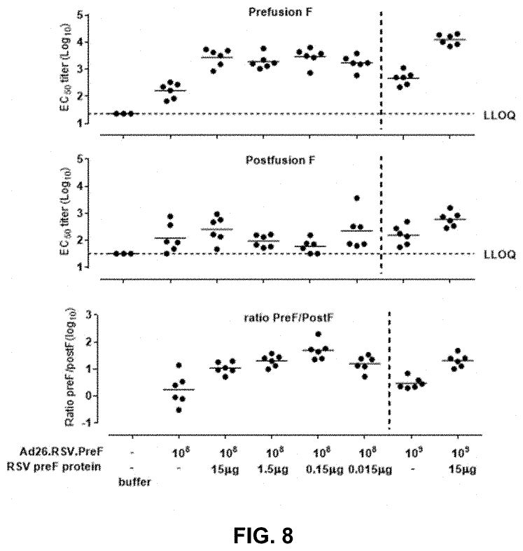

Cellular responses were measured in splenocytes taken 2 weeks post boost immunization. The number of IFNγ spot forming units (SFU) per 10⁶ splenocytes was determined by enzyme-linked immunospot (ELISPOT) assay. In FIG. 8, the geometric mean response per group is indicated with a horizontal line. The dashed line shows the limit of detection, defined as the 95% percentile of the SFU observed in non-stimulated splenocytes. Statistical analysis was performed with analysis of variance (ANOVA); ns=not significant.

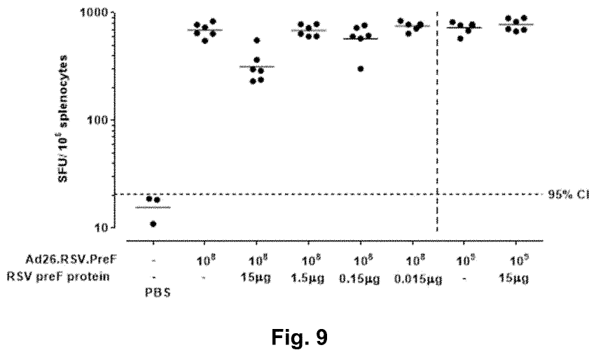

Prime and boost immunization with Ad26.RSV.pre-F mixed with 15, 1.5, 0.15 and 0.015 ug of RSV preF protein induced non-inferior ELISPOT IFNγ+ T cell responses compared with Ad26.RSV.pre-F alone (a 4-fold non-inferior margin, FIG. 9). The mix containing 1×10⁸ vp Ad26.RSV.pre-F 15 ug protein dose showed a tendency to being inferior compared with with Ad26.RSV.pre-F alone. A mixture of 10⁹ vp Ad26.RSV.pre-F with 15 ug showed non-inferior responses compared with 10⁹ vp Ad26.RSV.pre-F alone.

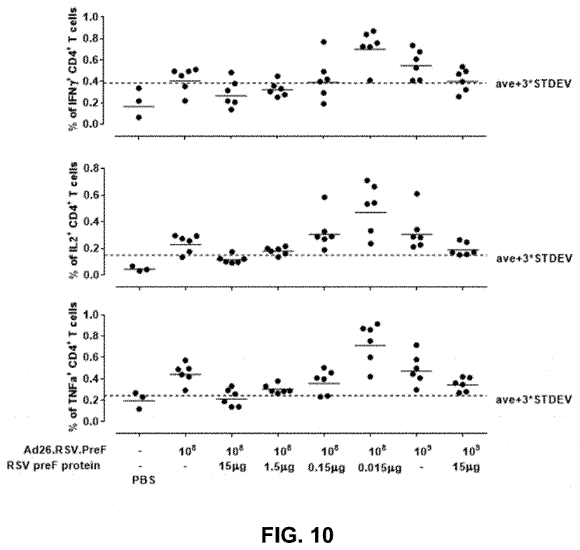

At 2 weeks post-boost immunization, animals were sacrificed, and splenocytes were isolated and stimulated with a peptide pool covering the RSV A2 F protein. The percentage of cytokine positive CD3+CD4+ and CD3+CD8+ splenocytes measured by intracellular cytokine staining (ICS) is shown in FIG. 10. The limit of detection (LOD) was defined as the mean background staining+3 standard deviations of medium controls. LOD CD3+CD4+ for IFNγ, TNFα and IL-2 were 0.39, 0.15 and 0.24, respectively and LOD CD3+CD8+ for IFNγ, TNFα and IL-2 were 0.19, 0.14 and 0.67, respectively. Statistical analysis was performed with analysis of variance (ANOVA); ns=not significant.

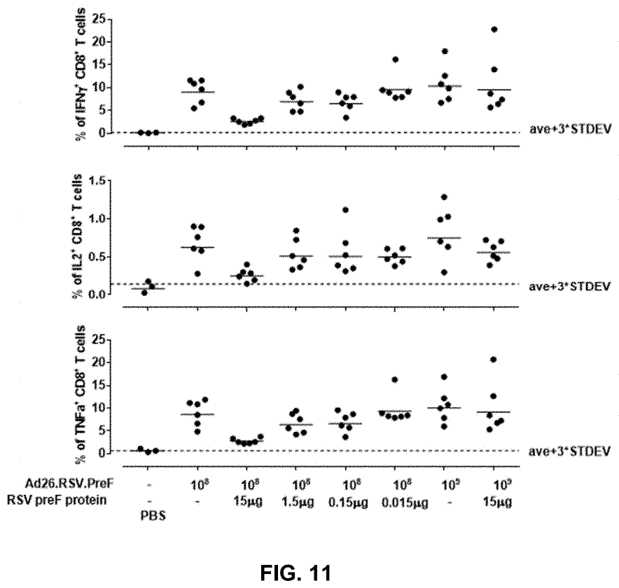

ICS revealed that 1×10⁸ vp Ad26.RSV.pre-F mixed with 15, 1.5, or 0.15 ug of RSV pre-F protein did not induce significantly different CD4+ IFNγ+, CD4+IL2+, and CD4+TNFα+ T cell responses compared with Ad26.RSV.preF alone, although there was a trend that 15 ug of RSV pre-F protein results in lower CD4+ T cell responses (ANOVA). Interestingly, 1×10⁸ vp Ad26.RSV.pre-F mixed with 0.015 ug of RSV pre-F protein showed significantly higher CD4+ IFNγ+, CD4+IL2+, and CD4+TNFα+ T cell responses compared with Ad26.RSV.preF alone. Ad26.RSV.preF (1×108 vp) mixed with 15, 1.5, 0.15, or 0.015 ug of RSV pre-F protein did not induce significantly different CD8+ IFNγ+, CD8+IL2+, or CD8+TNFα+ T cell responses compared with Ad26.RSV.preF alone (ANOVA) (FIG. 11).

### Example 3: Immunogenicity of RSV preF Protein and Ad26.RSV.preF in RSV Pre-Exposed Mice

In a prime-only study, Balb/c mice were pre-exposed to 5×10⁵ pfu RSV A2 via intranasal application 17 weeks prior to immunization. The mice then received a mixture of either 1.5 ug or 0.15 ug RSV pre-F protein together with 1×10⁸ or 1×10⁹ vp Ad26.RSV.pre-F (n=12 per group). Control groups received 1.5 ug RSV pre-F protein only (n=5), or 1×10⁸ or 1×10⁹ vp Ad26.RSV.pre-F only, or a mock immunization with formulation buffer mixture. Serum was taken 6 weeks post immunization.

Neutralizing Antibody Responses

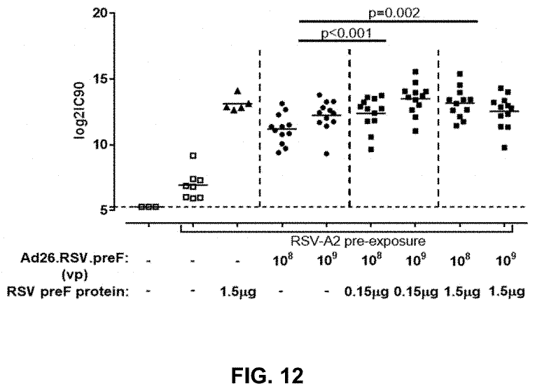

RSV CL57 virus neutralizing titers were determined using a firefly luciferase reporter-based assay. The IC90 titers are shown in FIG. 12. The mean response per group is indicated with a horizontal line. The dashed line shows the lower limit of quantification (LLOQ) of 5.28 log 2. Statistical analysis was performed with analysis of variance (ANOVA with Dunnet correction across Ad26.RSV.pre-F-dose comparison). The mock-immunized group showed that RSV A2 pre-exposed mice had VNT to RSV CL57 above the LLOQ for the assay. All immunization groups gave an increase in mean VNT compared with mock immunization. An across-Ad26.RSV.pre-F-dose comparison showed that immunization with a mixture of RSV pre-F protein and Ad26.RSV.pre-F gave higher VNT than Ad26.RSV.pre-F alone (0.15 ug RSV pre-F protein p<0.001; 1.5 ug RSV pre-F protein p=0.002, ANOVA for potentially censored measurements with Dunnett's correction for multiple comparisons).

### RSV Pre-F and Post-F Binding Antibody Responses

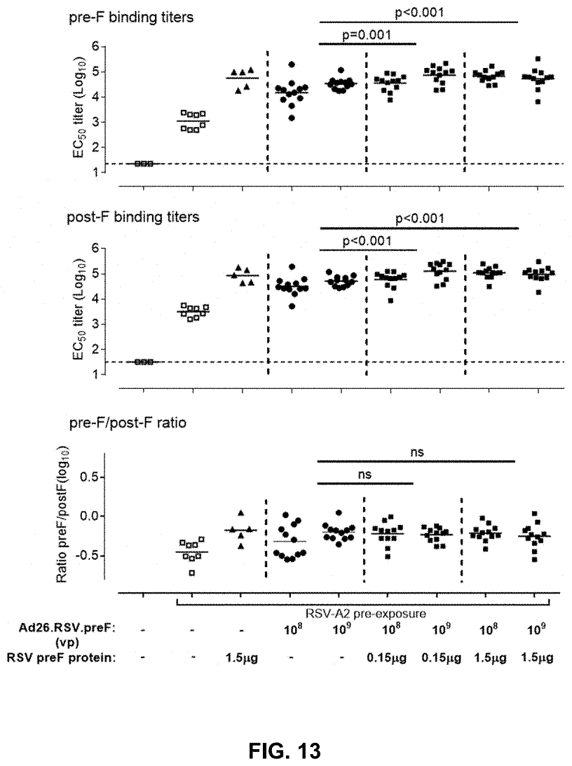

Serum was taken 6 weeks post immunization. IgG antibodies to RSV pre-F and RSV post-F were measured by ELISA. Plates were coated with anti-RSV F followed by addition of RSV pre-F or RSV post-F protein. The plates were incubated with serially diluted samples followed by detection with anti-mouse IgG, and the optical density was measured. In FIG. 13, pre-F and post-F binding antibody titers are given as the log 10 value of the EC50. The lower limit of quantification (LLoQ) is indicated with a dashed line. The lower graph displays the ratio between preF and postF antibodies for all samples that showed preF and postF titers above LLoQ. The mean response per group is indicated with a horizontal line.

Prior to immunization all RSV pre-exposed groups appeared to have comparable pre-F and post-F antibody titers (data not shown). After immunization, all groups had an increase in both pre-F and post-F antibody titers (FIG. 13). Mice immunized with a mixture of RSV pre-F protein and Ad26.RSV.pre-F had significantly higher pre-F and post-F titers compared with mice immunized with Ad26.RSV.pre-F alone (p≤0.001 for all groups, ANOVA for potentially censored observations across Ad26.RSV.pre-F dose with Dunnett's correction for multiple testing). The ratio of pre-F and post-F antibody titers was not significantly different between groups.

Cellular Responses

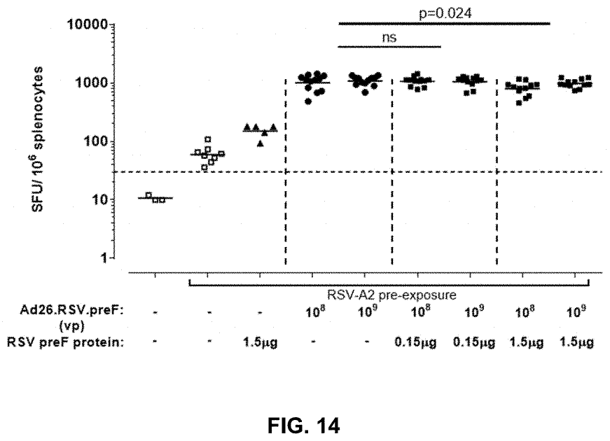

Splenocytes obtained 6 weeks after immunization were stimulated with a peptide pool covering the RSV A2 F protein. The number of IFNγ spot forming units (SFU) per 10⁶ splenocytes was determined by enzyme-linked immunospot (ELISPOT). The geometric mean response per group is indicated with a horizontal line (FIG. 14). The dashed line shows the limit of detection, defined as the 95% percentile of the SFU observed in non-stimulated splenocytes. Statistical analysis was performed with analysis of variance (ANOVA across Ad26.RSV.preF-dose comparison); ns=not significant.

The ELISPOT IFNγ SFU were not significantly different between the mixture of 0.15 ug RSV pre-F protein and Ad26.RSV.pre-F and Ad26.RSV.pre-F only (FIG. 14). A significantly lower response was observed with the mixture of 1.5 ug RSV pre-F protein and Ad26.RSV.pre-F compared with Ad26.RSV.pre-F only (p=0.024, ANOVA across-Ad26.RSV.pre-F-dose comparison). The difference was more pronounced for the lower (10⁸ vp) Ad26.RSV.preF dose than for the higher (10⁹ vp) dose. The cellular response was low in the group receiving RSV pre-F protein only.

The percentage of cytokine positive CD3+CD4+ and CD3+CD8+ splenocytes were measured by ICS. The limit of detection (LOD) was defined as the mean background staining +3 standard deviations of medium controls (FIG. 14). LOD CD3+CD4+ for IFNγ, TNFα and IL-2 were 0.30, 0.34 and 0.13, respectively. LOD CD3+CD8+ for IFNγ, TNFα and IL-2 were 0.65, 0.78 and 0.19, respectively. Statistical analysis was performed with a Cochran-Mantel-Haenszel test with Ad26.RSV.preF dose as stratification factor and with Bonferroni correction; ns=not significant.

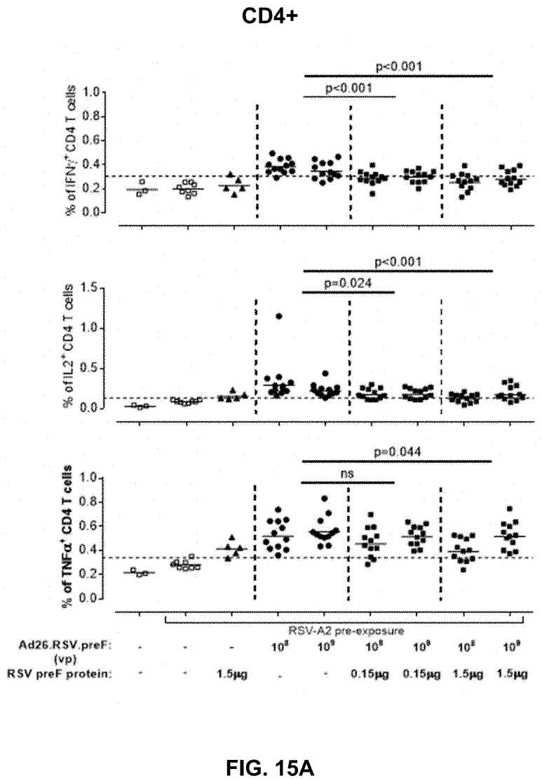

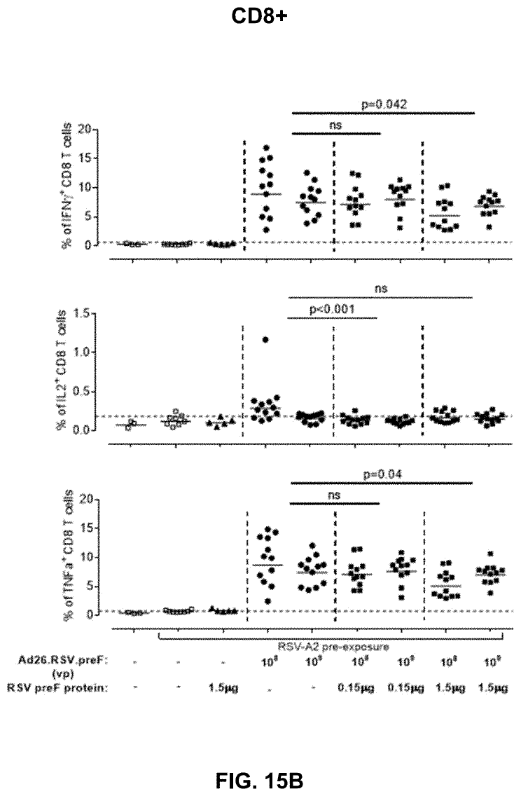

Pre-exposure only showed no detectable cytokine expression by CD4+ or CD8+ T cells (FIGS. 15A and B). Ad26.RSV.preF alone induced a low CD4+ T cell response (IFNγ, IL-2 and TNFα expressing CD4+ T cells, mostly below 1% of CD3+CD4+ cells). A mix of Ad26.RSV.preF and PRPM showed a significantly lower IFNγ, IL-2 and TNFα responses for both concentrations of PRPM in the mix, with the exception of 0.15 ug for CD4+TNFα+ T cells (FIG. 15A). Ad26.RSV.preF alone induced CD8+ T cells expressing IFNγ, IL-2 (low percentage) and TNFα (FIG. 15B). In line with the ELISPOT results, a mix of Ad26.RSV.preF and 1.5 ug PRPM induced significantly lower IFNγ and TNFα responses compared with mice receiving Ad26.RSV.preF alone (p=0.042 and 0.040, respectively, CMH test across Ad dose, FIG. 15B). The IL-2 response was also reduced in mice receiving a mix of Ad26.RSV.preF and 0.15 ug PRPM (p<0.001).

These data show that the Ad26.RSV.preF component induces cellular responses and indicate that addition of RSV preF protein may impact the cellular response depending on the RSV preF protein/Ad26.RSV.preF ratio used.

### Example 4: Immunogenicity of Heterologous Regimens of RSV preF Protein and Ad26.RSV.preF in RSV Pre-Exposed Mice

Immunogenicity of a mixture of RSV pre-F protein and Ad26.RSV.pre-F after prime-only immunization was compared with a heterologous Ad26.RSV.pre-F prime, RSV pre-F protein boost regimen in mice. Balb/c mice were pre-exposed to 5×10⁵ pfu RSV A2 intranasal application, and 26 weeks later received a prime immunization with a mixture of 0.15 ug RSV pre-F protein and 1×108 vp Ad26.RSV.pre-F (n=13), or 1×10⁸ vp Ad26.RSV.pre-F only (n=12). Prime-boost groups with a 4 week dosing interval were immunized with 1×10⁸ vp Ad26.RSV.pre-F prime and 0.15 ug RSV pre-F protein boost (n=12) or 0.15 ug RSV pre-F protein prime and boost (n=4). The mock group received formulation buffer (n=7).

Neutralizing Antibody Responses

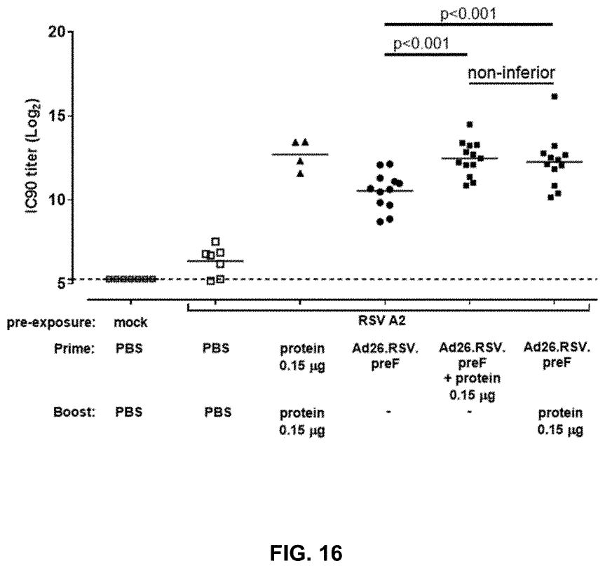

Serum was taken 6 weeks post-prime (2 weeks post boost) immunization. RSV CL57 virus neutralizing titers were determined using a firefly luciferase reporter based assay. The mean response per group is indicated with a horizontal line. The dashed line shows the lower limit of quantification of 5.28 log 2. Statistical analysis was performed with analysis of variance (ANOVA) and non-inferiority testing. The non-inferiority margin was set as a 4-fold change in IC90 titer, ie 2 log 2. A robust neutralizing antibody response against the RSV CL57 strain was seen 6 weeks after single immunization with a mixture of 0.15 ug RSV pre-F protein and 1×10⁸ vp Ad26.RSV.pre-F, which was non-inferior to the heterologous Ad26.RSV.preF prime, RSV pre-F protein boost regimen using the same doses (FIG. 16). The heterologous prime-boost regimen also induced a significantly higher VNT compared with single immunization with Ad26.RSV.pre-F alone (p<0.001, ANOVA).

RSV Pre-F and Post-F Binding Antibody Responses

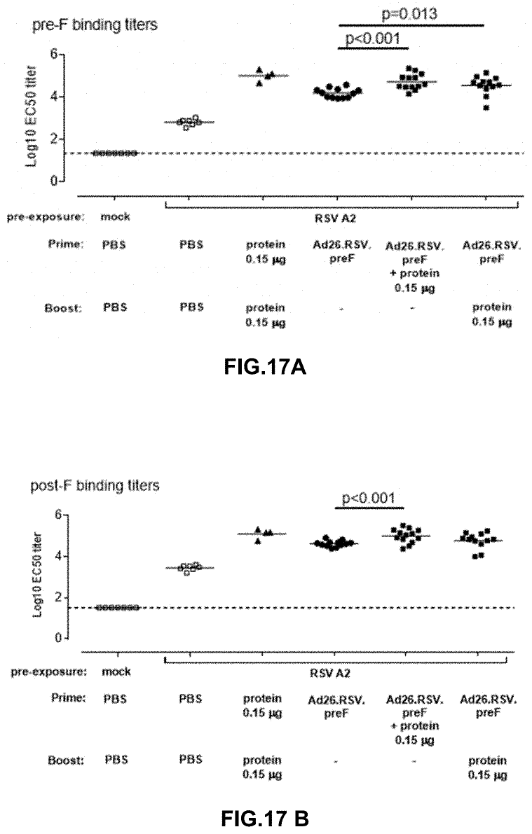

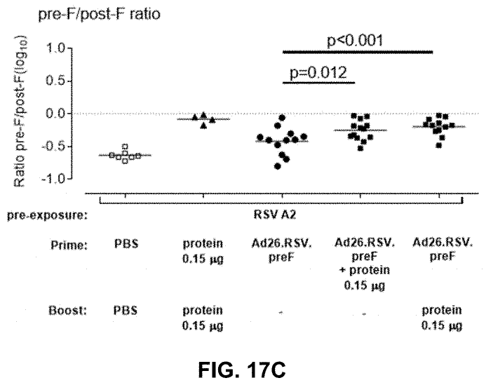

Two weeks post boost (Week 6), the mixture of RSV preF protein and Ad26.RSV.preF showed non-inferior pre-F and post-F antibody titers compared with mice receiving the heterologous Ad26.RSV.pre-F prime, RSV pre-F protein boost regimen (FIGS. 17A and B). The heterologous prime-boost regimen induced significantly higher pre-F antibody titers (p=0.013) and ratio of pre-F/post-F titers (p<0.001) compared with Ad26.RSV.pre-F prime only (FIGS. 17A and C); the post-F titers (FIG. 17B) were similar between these groups. It should be noted that before boost (Week 4), the two groups receiving Ad26.RSV.preF prime showed significantly different levels of pre-F and post-F titers (p=0.009 and p=0.006 respectively, ANOVA), probably by chance. Exploratory analysis showed that the group immunized with a mixture of RSV preF protein and Ad26.RSV.preF showed significantly higher pre-F and post-F antibody titers compared with mice receiving Ad26.RSV.preF alone, at both Week 4 and Week 6 (p<0.001 for all comparisons, ANOVA). At Week 6, mice receiving Ad26.RSV.preF alone had a significantly lower pre-F/post-F antibody ratio than mice receiving the mixture of RSV preF protein and Ad26.RSV.preF (p=0.012, ANOVA).

Cellular Responses

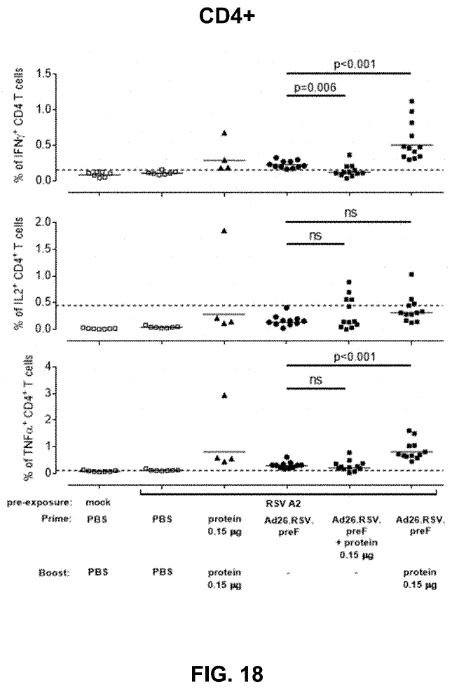

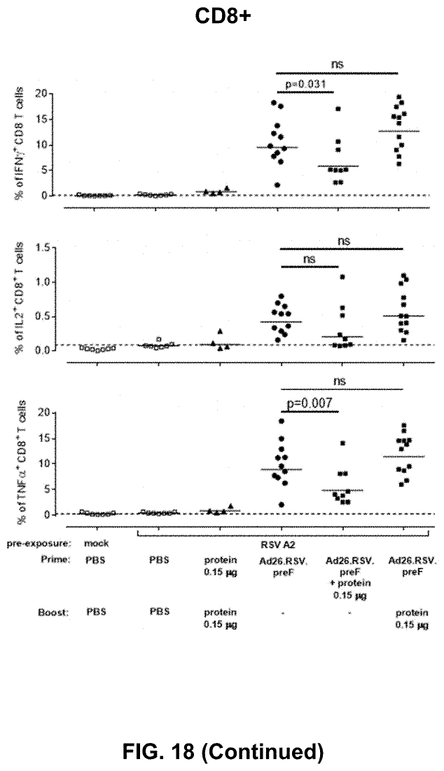

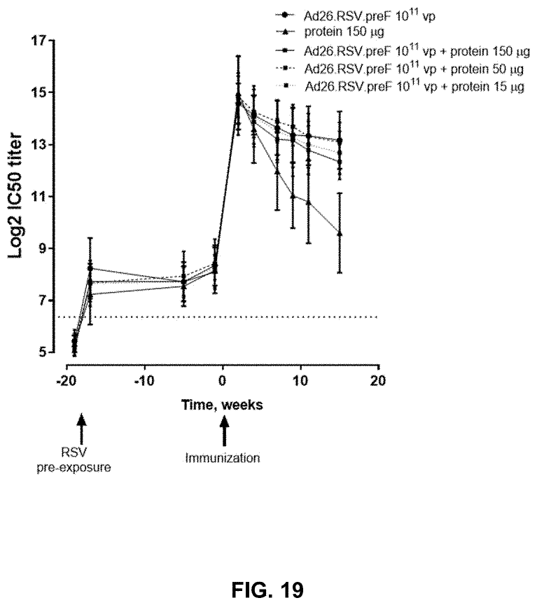

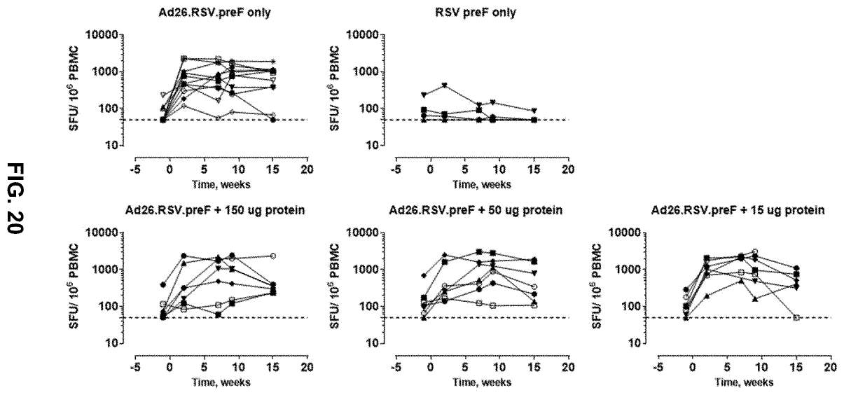

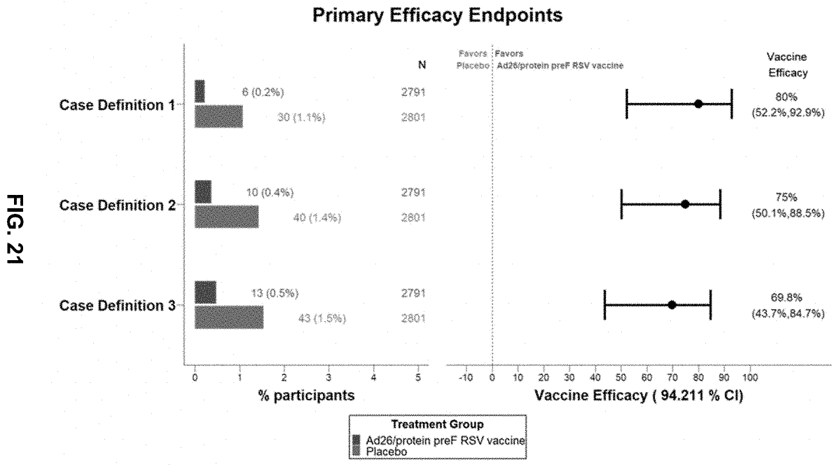

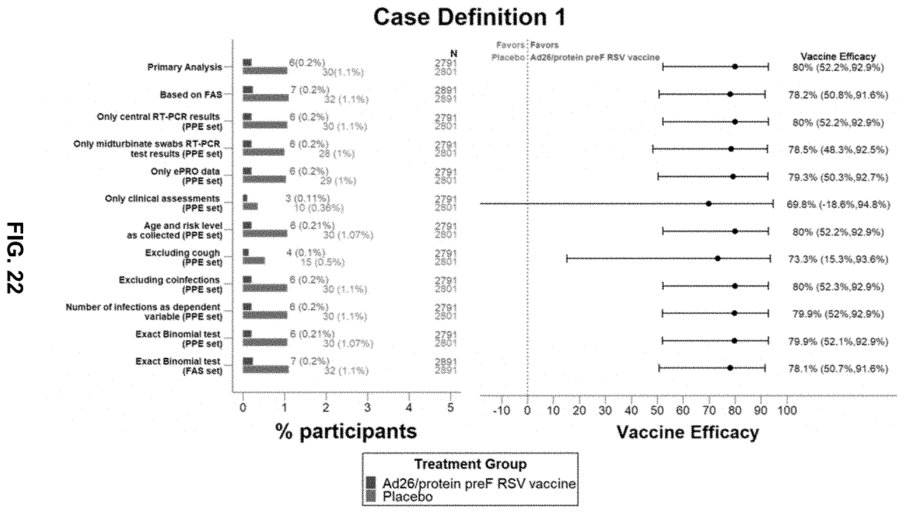

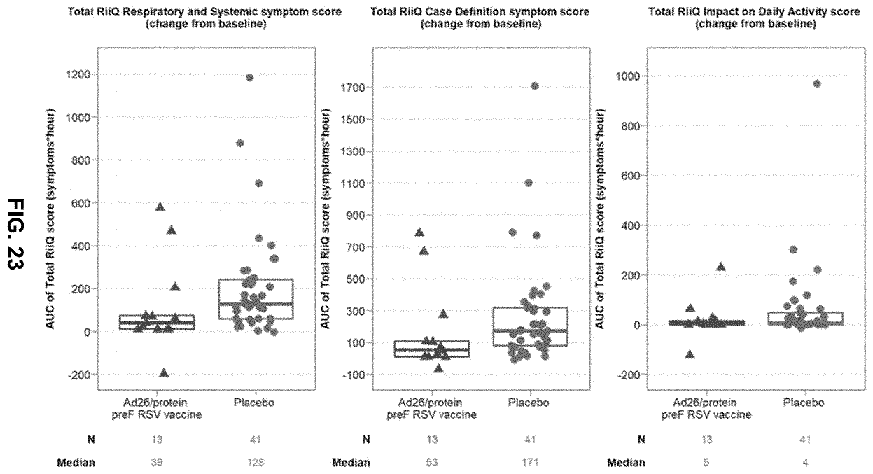

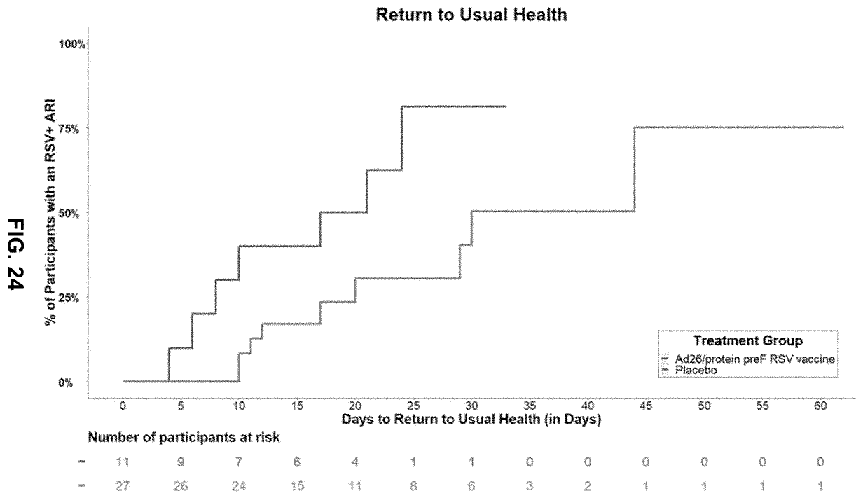

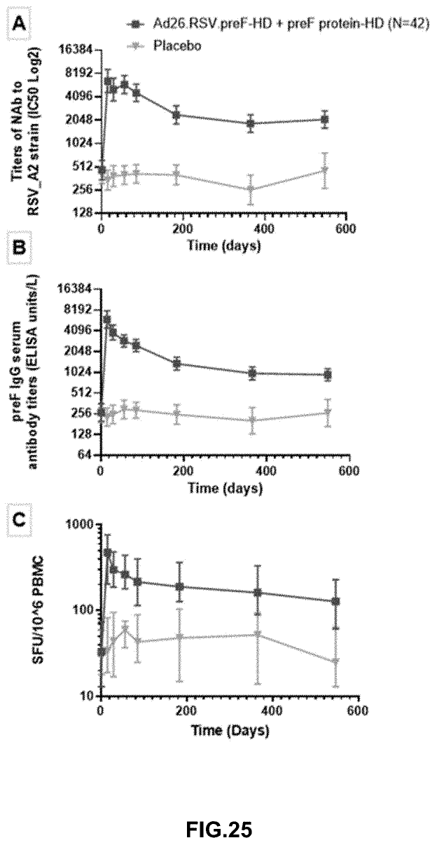

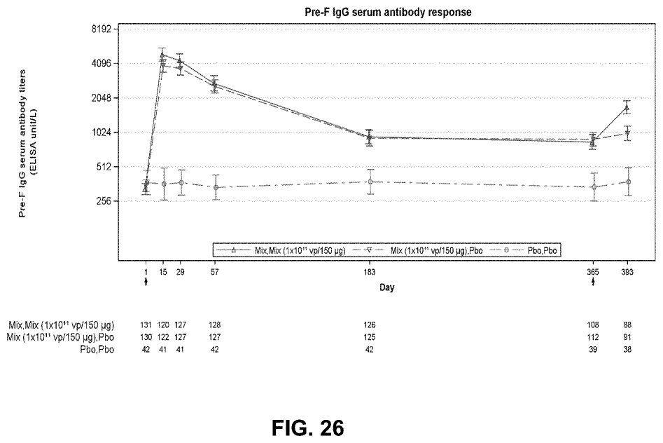

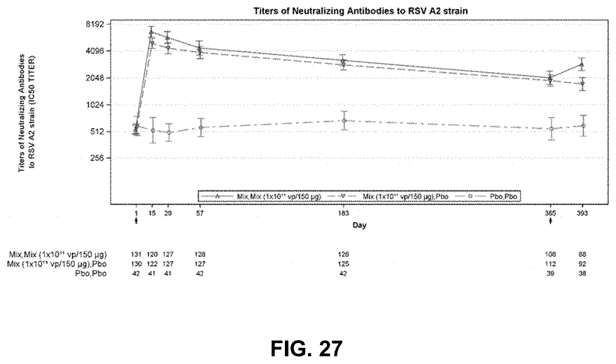

The cellular response was measured by IFNγ ELISPOT and ICS for IFNγ, TL-2 and TNFα. Due to technical failure in the ELISPOT assay, no conclusions can be drawn from that assay. In the ICS assay, the heterologous Ad26.RSV.preF prime, RSV preF protein boost regimen induced significantly higher CD4+ T cell TNFα and IFNγ responses compared with Ad26.RSV.preF alone (both p<0.001, ANOVA) (FIG. 18). The mixture of 0.15 ug RSV preF protein and 1×10⁸ vp Ad26.RSV.preF induced significantly lower CD8+ IFNγ, CD8+TNFα and CD4+ IFNγ T cell responses compared with Ad26.RSV.preF alone (p<0.05 for all, ANOVA).

### Example 5: Immunogenicity of RSV preF Protein and Ad26.RSV.preF in RSV Pre-Exposed Non-Human Primates (NHP)

African Green Monkeys (females, 9-26 y) were intranasally pre-exposed with 7.5×10⁵ pfu RSV Memphis 37 strain. Successful pre-exposure was confirmed by RSV post-F ELISA of serum samples obtained 14 weeks later (data not shown). The monkeys were then allocated to the study groups based on RSV post-F ELISA titers and age to give an even distribution in RSV pre-exposure antibody titers between the groups. Nineteen weeks after pre-exposure, the animals received a single immunization with 10¹¹ vp Ad26.RSV.preF, 150 ug RSV preF protein or with a mixture of 10¹¹ vp Ad26.RSV.preF and 150 ug, 50 ug or 15 ug RSV preF protein, respectively.

Neutralizing Antibody Responses

The RSV pre-exposed NHP had VNT against RSV CL57 above the limit of detection 1 week before immunization. An increase in VNT was observed in all vaccine groups 2 weeks after immunization (FIG. 19). No significant differences in VNT were observed between the group receiving Ad26.RSV.preF only and the groups receiving the mixture of Ad26.RSV.preF and RSV preF protein, at any time point tested (ANOVA with Dunnett correction for multiple testing). The VNT response was very high in the Ad26.RSV.preF immunized group, and therefore it was not possible to conclude on the additional value of RSV preF protein in the mixture in this model.

The VNT response to RSV preF protein appeared to be less durable than immunization with Ad26.RSV.preF. Animals receiving 150 ug RSV preF protein did not show a significantly different VNT compared with animals receiving Ad26.RSV.preF or a mixture of 150 ug RSV preF protein and Ad26.RSV.preF 2 and 4 weeks after immunization. However, 7, 9, 11 and 15 weeks after immunization, the VNT induced by RSV preF protein were significantly lower compared with Ad26.RSV.preF only, and were also lower at 9, 11, and 15 weeks compared with the mixture of 150 ug RSV preF protein and Ad26.RSV.preF (p<0.05 for all, ANOVA with Dunnett's correction for multiple testing).

Cellular Responses

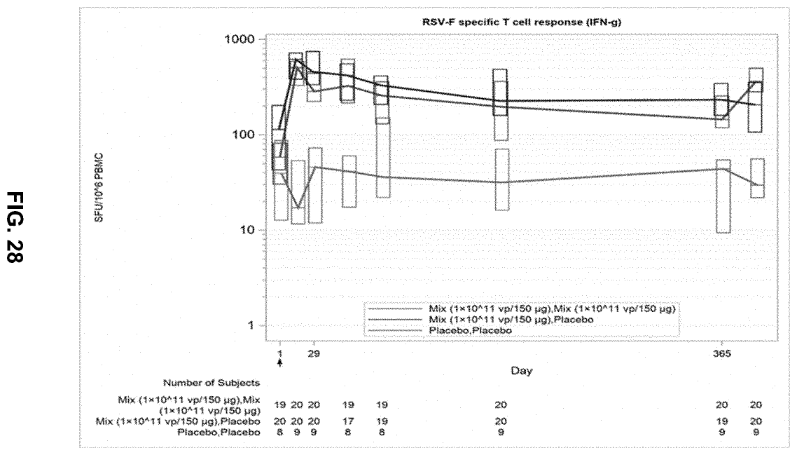

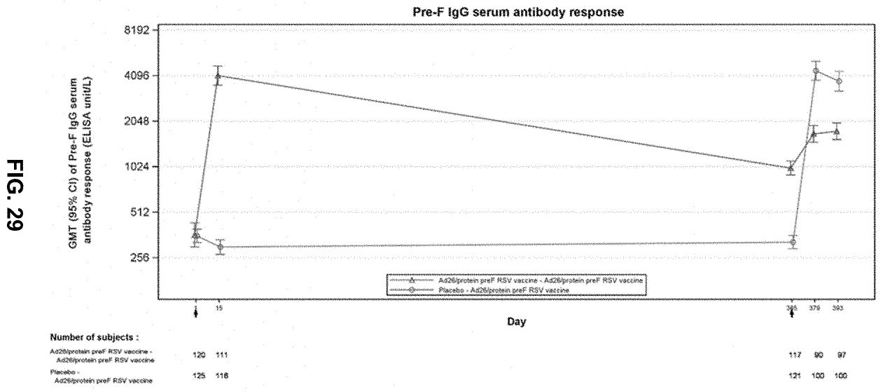

RSV F-specific T cell responses prior to vaccination were generally low across groups in most animals. There was a large variation in the RSV F specific cellular response between the individual animals (FIG. 20). Comparing with cellular responses before immunization, animals immunized with Ad26.RSV.preF alone showed significantly higher responses at week 7 and 9 (p=0.03 and 0.02, respectively, ANOVA with Bonferroni correction for multiple comparisons). Furthermore, a mix with 50 ug RSV preF protein showed a significantly higher response at all time points (p=0.03, 0.04, 0.04, 0.04 for week 2, 7, 9, and 15, respectively) and a mix with 15 ug RSV preF protein showed a significantly higher response at week 2 and 9 (p=0.0003 and p=0.0001, respectively). Immunization with a mix of Ad26.RSV.preF and 150 ug RSV preF protein or 150 ug RSV preF protein alone did not show a significant increase in T cell responses at any time point tested. No significant differences were observed between the group receiving Ad26.RSV.preF only and the groups receiving Ad26.RSV.preF and RSV preF protein combination, at any time point tested (ANOVA with Dunnett's correction for multiple testing). Animals immunized with Ad26.RSV.preF only and with a mix of Ad26.RSV.preF and 150 ug RSV preF protein showed a significantly higher cellular response compared with animals immunized with 150 ug RSV preF protein alone at all time points tested (p≤0.05 for all).

### Example 6: Phase 2b Study to Assess the Efficacy, Immunogenicity and Safety of an Ad26.RSV.preF-Based Regimen in the Prevention of RT-PCR-Confirmed RSV-Mediated Lower Respiratory Tract Disease in Adults Aged 65 Years and Older

A multi-center, randomized, double-blind, placebo-controlled Phase 2b proof-of-concept study in male and female participants aged ≥65 years who are in stable health was performed. A target of up to 5,800 participants was to be enrolled. A schematic overview of the study design and groups is depicted below.

| Group | N^{a} | Day 1 |
|---|---|---|
| Group 1 | 2,900 | Ad26.RSV.preF (1 × 10¹¹ vp)/ |
| | | RSV preF protein (150 μg) |
| Group 2 | 2,900 | Placebo |

Randomization: Participants are randomized in parallel in a 1:1 ratio to 1 of 2 groups to receive Ad26.RSV.preF/RSV preF protein vaccine or placebo. The randomization will be stratified by age categories (65-74 years, 75-84 years, ≥85 years) and by being at increased risk for severe RSV disease (yes/no), and done in blocks to ensure balance across arms.

Vaccination schedules/Study duration: Screening for eligible participants will be performed pre-vaccination on Day 1. Participants will be followed up until the end of the RSV season. If the study continues beyond the first RSV season (conditional on Primary Analysis results), the study duration is approximately 1.6 years.

Primary analysis set for efficacy: The Per-protocol Efficacy (PPE) population will include all randomized and vaccinated participants excluding participants with major protocol deviations expecting to impact the efficacy outcomes. Any participant with an RT-PCR-confirmed RSV-mediated ARI with onset within 14 days after vaccination will be excluded, as well as participants who discontinue within 14 days after vaccination.

Primary efficacy endpoint: The three primary efficacy endpoints are first occurrence of RT-PCR confirmed RSV-mediated LRTD according to each of the 3 case definitions shown in the table below:

| Case Definition #1 | Case Definition #2 | Case Definition #3 |
|---|---|---|
| ≥3 symptoms of LRTI | ≥2 symptoms of LRTI | ≥2 symptoms of LRTI, |
| (new onset or | (new onset or | OR |
| worsening) | worsening) | ≥1 symptom of LRTI |
| | | combined with |
| | | ≥1 systemic symptom |
| | | (new onset or |
| | | worsening) |

+RT-PCR confirmation of RSV
LRTI = lower respiratory tract infection
Symptoms are collected via the RiiQ, an ePRO questionnaire completed by the participant at baseline and daily during the ARI, and via a clinical assessment by the PI completed at baseline and at the day 3-5 visit during the ARI.
First occurrence of a considered endpoint is defined as the first day of symptoms of the first RSV-confirmed ARI episode where the criteria for the respective case definition are fulfilled on at least one assessment of the considered episode.

The 3 case definitions assessed in this study were designed to cover a range of RSV disease severity. The presence of a combination of 3 symptoms of lower respiratory tract infection similar to those used in this study have been associated with a 3-fold higher risk of a severe outcome (Belongia et al., Adult RSV Epidemiology and Outcomes, OFID, 2018).

### Primary Objective(s):

To demonstrate the efficacy of active study vaccine in the prevention of reverse transcriptase polymerase chain reaction (RT PCR)-confirmed RSV-mediated lower respiratory tract disease (LRTD) according to one of the three case definitions, when compared to placebo.

### Vaccine:

The active study vaccine was an Ad26.RSV.preF/RSV preF protein mixture, comprising:
Ad26.RSV.preF, a replication-incompetent adenovirus serotype 26 (Ad26) containing a deoxyribonucleic acid (DNA) transgene that encodes the pre-fusion conformation-stabilized F protein (pre-F) derived from the RSV A2 strain, i.e. the pre-fusion conformation-stabilized F protein (pre-F) of SEQ ID NO: 5; and
RSV preF protein, a pre-fusion conformation-stabilized F protein derived from the RSV A2 strain, i.e. the RSV preF protein of SEQ ID NO: 6 or 7.
   The vaccine was administered as a single injection in the deltoid muscle. All injections are 1 mL in volume.

The following doses were administered:
Ad26.RSV.preF was supplied at a concentration of 2×10¹¹ vp (viral particles)/1 mL in single-use vials. Dose levels of 1×10¹¹ vp are used.
RSV preF protein was supplied at a concentration of 0.3 mg/l mL in single-use vials. Dose levels of 150 μg are used.
Placebo for Ad26.RSV.preF, and RSV preF protein.
   Serious adverse events (SAEs) were reported from administration of study vaccine until the end of the RSV season, or 6 months after.

### Summary of Results

Below, the topline results of the primary analysis are described. Unblinded results are presented. Data up to May 15, 2020 are included. This was the date when all participants were expected to have completed their End of Season call or had discontinued earlier. One clinical site was unable to collect end of season data including SAEs prior to the database cutoff due to the COVID-19 pandemic. Additionally, due to the increasing incidence of COVID-19 cases in the US, the ARI surveillance period was shortened from 30 Apr. 2020 to 20 Mar. 2020.

Solicited AEs (up to 7 days post-vaccination) and unsolicited AEs (up to 28 days post-vaccination) were captured in a subset of ˜700 participants (the Safety Subset). SAEs were captured in all participants. Humoral and cellular immunogenicity over time was collected for a subset of 200 participants (the Immuno Subset).

The study is considered successful as soon as vaccine efficacy (VE) is demonstrated for at least one of the primary endpoints. To control the false positive rate for multiplicity, the Spiessens and Debois method is applied. If the p-value is below the multiplicity corrected alpha level for at least 1 of the 3 primary endpoints, proof of concept is demonstrated. Correspondingly, if the multiplicity corrected confidence interval (CI) is above 0 for at least 1 of the 3 primary endpoints, the study is successful.

A total of 6673 participants were screened across 40 sites in the US. Of those, 857 were screening failures, 34 were randomized not vaccinated and 5782 participants were randomized and vaccinated (2891 in each group). 107 (3.7%) participants in the active group and 100 (3.5%) participants in the placebo group discontinued the study, the majority (129 participants) withdrew consent. All other participants were still ongoing at the time of database cut-off. In the full analysis (FA) set, 57.7% of the participants were female and 92.5% were white. The median age was 71 years, ranging from 65 to 98 years. The median BMI was 28.7 kg/m², ranging from 11.7 to 41.1 kg/m². 25.4% of the participants was at increased risk for RSV disease (risk level as collected in eCRF, using CDC guidance (i.e. chronic heart and lung disease)) and 26.2% of the participants was pre-frail or frail at baseline. 92 (3.2%) participants in the Ad26/protein preF RSV vaccine group and 83 (2.9%) in the placebo group, had a major protocol deviation impacting efficacy. Those participants were excluded from the Per Protocol Efficacy (PPE) set, the primary analysis set for efficacy analyses.

### Primary Endpoint Analysis

The three primary efficacy endpoints are first occurrence of RT-PCR confirmed RSV-mediated LRTD according to each of the three Case Definitions as described above.

Symptoms were collected via the RiiQ, an ePRO questionnaire completed by the participant at baseline and daily during the ARI (acute respiratory infection), and via a clinical assessment by the PI completed at baseline and at the day 3-5 visit during the ARI. Signs and symptoms taken into account for the determination of Case Definitions are shown in Table 1. Counting of the number of symptoms with new onset or worsening was done per day and per assessment, so clinical assessment or patient reported outcome in the eDiary or in the eDevice was not combined for the counting.

TABLE 1
Symptoms of Lower Respiratory Tract Infection and Systemic
Symptoms as per RiiQ or Clinical Assessment

| | | | Clinical Assessment |
|---|---|---|---|
| | Symptoms from | | Term (ARI Days 3- |
| | Case Definition | RiiQ Term | 5 Clinical Visit) |
| Symptoms | Cough | Cough | Cough |
| of LRTI | Shortness of | Short of breath | Dyspnea or |
| | breath | | decreased oxygen |
| | | | saturation |
| | Sputum | Coughing up | Sputum production |
| | production | phlegm (sputum) | |
| | Wheezing | Wheezing | Wheezing or rhonchi, |
| | | | rales or other sign of |
| | | | consolidation |
| | Tachypnea | | Tachypnea |
| Systemic | Fatigue | Fatigue | Malaise (tiredness) |
| Symptoms | Fever | (tiredness) | Fever |
| | Feverishness | Feeling | |
| | | feverish or | |
| | | Fever* | |

LRTI = lower respiratory tract infection,
RiiQ = Respiratory Infection Intensity and Impact Questionnaire
*Fever defined based on the daily temperature reported from the participants in the eDiary

First occurrence of a considered endpoint is defined as the first day of symptoms of the first RSV-confirmed ARI episode where the criteria for the respective Case Definition are fulfilled on at least one assessment of the considered episode. Only episodes occurring in the first season of the participant are taken into account for the primary analysis.
For each of the 3 primary endpoints the following is performed: an exact Poisson regression will be fitted with the event rate, defined as the number of cases over the follow-up time (offset) as dependent variable and the vaccination group, age and being at increased risk for severe RSV disease (both as stratified) as independent variables.
The primary analysis set for efficacy is the PPE set which includes all randomized and vaccinated participants excluding participants with major protocol deviations expecting to impact the efficacy outcomes. Any participant with an RT-PCR-confirmed RSV-mediated ARI with onset within 14 days after vaccination will be excluded, as well as participants who discontinue within 14 days after vaccination.
The study was successful as soon as vaccine efficacy (VE) is demonstrated for at least one of the primary endpoints. To control the false positive rate for multiplicity, the Spiessens and Debois method is applied. The exact one-sided p-value, from the Poisson regression described above, corresponding to vaccination group will be compared with the multiplicity corrected alpha level. If the p-value is below the cut-off for at least one of the three primary endpoints, proof of concept is demonstrated. Correspondingly, if the multiplicity corrected confidence interval (CI) is above 0 for at least one of the three primary endpoints, the study is successful.

### Primary Efficacy Analysis

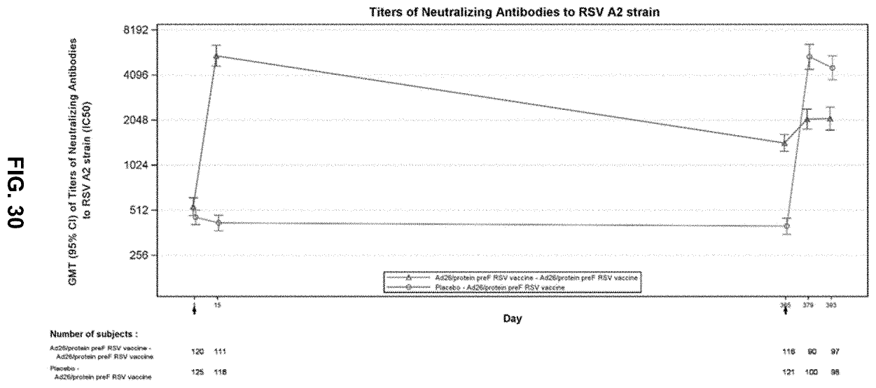

The primary analysis results are shown in Table 2 and FIG. 21. Significance is shown for all three primary endpoints.

TABLE 2
Primary Efficacy Analysis: Percentage of participants with RT-PCR confirmed RSV-
mediated LRTD according to each of the 3 Case Definitions and Vaccine Efficacy
of their first occurrence; Per Protocol Efficacy set (study VAC18193RSV2001)

| | Ad26/protein | | | | | | | |
|---|---|---|---|---|---|---|---|---|
| | preF | | | | Vaccine | | | |
| | RSV vaccine | | Placebo | | Efficacy | | | |
| | n (%) | | n (%) | | (94.211% CI) | | p-value | α level |
| Analysis Set: Per | 2791 | | 2801 | | | | | |
| Protocol Efficacy Set | | | | | | | | |
| Case Definition 1 | 6 | (0.2%) | 30 | (1.1%) | 80.0 | (52.2, 92.9) | 0.00004 | 0.02895 |
| Case Definition 2 | 10 | (0.4%) | 40 | (1.4%) | 75.0 | (50.1, 88.5) | 0.00001 | 0.02895 |
| Case Definition 3 | 13 | (0.5%) | 43 | (1.5%) | 69.8 | (43.7, 84.7) | 0.00004 | 0.02895 |

Case Definition 1: ≥3 symptoms of LRTI + RT-PCR confirmation for RSV
Case Definition 2: ≥2 symptoms of LRTI + RT-PCR confirmation for RSV
Case Definition 3: ≥2 symptoms of LRTI, OR ≥1 symptom of LRTI combined with ≥1 systemic symptom + RT-PCR confirmation for RSV
The p-value and the Vaccine efficacy are calculated based on an exact Poisson regression with the event rate, defined as the number of cases over the follow-up time (offset) as dependent variable and the vaccination group and age and being at increased risk for severe RSV ARI (both as stratified) as independent variables.
The α level is adjusted to account for the multiple endpoints.
All subject data up to May 15, 2020 are included

### Sensitivity Analyses

Several sensitivity analyses were performed. Each sensitivity analysis is modifying one of the specifications used for the primary analysis (population, model, dependent variable, independent variables, . . . ).

The results of the sensitivity analyses are presented in FIG. 22 for Case Definition 1. In general, the sensitivity analyses are in line with the primary analysis results: point estimates and confidence intervals are similar, except for the sensitivity analysis for CD1 using only clinical assessments (lower bound VE below 0%), and for CD1 excluding cough (lower bound VE of 15.3%), which might be explained by the low number of events observed. For CD2 and CD3, more events are observed for the sensitivity analyses using only clinical assessments and excluding cough and the results are in line with the primary analysis results for those Case Definitions.

### Patient Reported Outcomes

### RiiQ (Respiratory Infection Intensity and Impact Questionnaire)

Participants were asked whether during the past 24 hours, they had any of the following symptoms: cough, sore throat, headache, nasal congestion, feeling feverish, body aches and pains, fatigue, neck pain, interrupted sleep, coughing up phlegm (sputum), short of breath or loss of appetite.

In the RiiQ Symptom Scale each symptom was rated on the following scale: 0=None, 1=Mild, 2=Moderate, and 3=Severe. Based on this questionnaire, total scores over time were calculated:
Total RiiQ Respiratory and Systemic symptom score is per timepoint assessed as the mean of all symptom scores (2 URTI symptoms, 4 LRTI symptoms and 7 systemic symptoms).
Total RiiQ Case Definition symptom score is per timepoint assessed as the mean of 4 LRTI symptoms (Cough, Wheezing, Shortness of breath, and Coughing up phlegm/sputum) and 2 systemic symptoms used in the Case Definitions, fatigue and feeling feverish

The RiiQ Impact on Daily Activity scale (question 2, Attachment 1) consists of 7 activities. Ability to perform each activity item is rated on the following scale: 0=No difficulty, 1=Some difficulty, 2=Moderate Difficulty, and 3=Great difficulty. The total RiiQ Impact on Daily Activity score is calculated as the mean of all 7 items (range 0-3).

For the above scores obtained during the RT-PCR confirmed RSV ARIs, AUC are calculated and presented with boxplots in FIG. 23. The figures show that in participants with an RT-PCR confirmed RSV ARI, the median (Q1; Q3) AUC of the total RiiQ respiratory and systemic symptom score was 39 (11; 74) in the Ad26/protein preF RSV vaccine group, compared to 128 (58; 242) in the Placebo group. For the AUC of the total RiiQ symptom score for symptoms included in the CDs (RiiQ CD score), the medians (Q1; Q3) were 53 (10; 108) and 171 (79; 317) respectively. For the RiiQ impact on daily activity score, the median (Q1; Q3) AUCs were 5 (0; 13) and 4 (0; 48). Lower AUCs indicate less severe disease (i.e. symptoms more comparable to baseline symptoms). These findings support that, when infected with RSV, subjects who received the Ad26/protein preF vaccine have less severe symptoms compared to subjects who received the placebo.

### Patient Global Impression (PGI) Scores

The PGI questionnaire was collected daily during the ARI and is used to evaluate the overall health of the participants.

Participants were asked whether they had returned to their usual health after developing symptoms suggesting an ARI. A Kaplan-Meier of the number of days a participant took to return to its usual health is shown in FIG. 24. Importantly, these data show that participants in the Ad26/protein preF RSV vaccine group tend to return to their usual health more rapidly compared to placebo recipients, highlighting the positive impact of the vaccine on the course of RSV disease (median time to return to usual health: Ad26/protein RSV vaccine group: 19 days; placebo: 30 days).

### Immunogenicity

Humoral and cellular immunogenicity over time was collected for a subset of 200 participants (the Immuno Subset). The randomisation ratio in the Immunosubset was also 1:1. Table 4 provides a summary of the immunogenicity observed in the Ad26/protein preF RSV vaccine group. The analysis was performed on the Per Protocol Immunogenicity Set.

TABLE 4
Overview of immunogenicity; Per Protocol Immunogenicity Set

| | Ad26/protein preF RSV vaccine (N = 97) | | | | | |
|---|---|---|---|---|---|---|
| Assay | Baseline | | Day 15 | | Day 169 | |
| VNA A2 GMT (95% CI) | 542 | (457; 643) | 7244 | (5889; 8912) | 3057 | (2523; 3703) |
| VNA B GMT (95% CI) | 4079 | (3501; 4752) | 38006 | (31693; 45577) | 17362 | (14768; 20413) |
| ELISpot Median (Q1, Q3) | 34 | (34; 76) | 444 | (279; 641) | 201 | (123; 324) |

The vaccine of the invention thus induced a robust and long lasting humoral and cellular immune response.

### Safety

Solicited AEs (up to 7 days post-vaccination) and unsolicited AEs (up to 28 days post-vaccination) were captured in a subset of ˜700 participants (the Safety Subset). SAEs were captured in all participants. Table 5 provides an overview of the safety reported in the locked database.

In the total population up to database cut-off, there are 132 (4.6%) and 136 (4.7%) participants that experienced at least one serious adverse event in the Ad26/protein preF RSV vaccine group and Placebo group, respectively. There are no deaths and serious adverse events considered related to the vaccination by the investigator.

TABLE 5
Summary of Safety; Full Analysis set

| | Ad26/protein | | | |
|---|---|---|---|---|
| | preF | | | |
| | RSV vaccine | | Placebo | |
| | n (%) | | n (%) | |
| Solicited AEs (Safety Subset, 7 days | N = 348 | | N = 347 | |
| post-vaccination): | | | | |
| Participants with 1 or more: | | | | |
| Solicited AEs | 179 | (51.4%) | 70 | (20.2%) |
| Solicited AEs of at least grade 3 | 11 | (3.2%) | 2 | (0.6%) |
| Solicited local AEs | 132 | (37.9%) | 29 | (8.4%) |
| Solicited local AEs of at least grade 3 | 6 | (1.7%) | 1 | (0.3%) |
| Solicited systemic AEs | 144 | (41.4%) | 57 | (16.4%) |
| Solicited systemic AEs of at least | 7 | (2.0%) | 1 | (0.3%) |
| grade 3 | | | | |
| Solicited AEs (Safety Subset, 28 days | N = 348 | | N = 347 | |
| post-vaccination): | | | | |
| Participants with 1 or more: | | | | |
| Unsolicited AEs | 58 | (16.7%) | 50 | (14.4%) |
| Unsolicited AEs of at least grade 3 | 6 | (1.7%) | 5 | (1.4%) |
| Unsolicited AEs thought to be related | 18 | (5.2%) | 8 | (2.3%) |
| to study vaccine | | | | |
| SAEs and AEs leading to | N = 2891 | | N = 2891 | |
| discontinuation (all participants, | | | | |
| whole study) | | | | |
| Participants with 1 or more: | | | | |
| SAEs | 132 | (4.6%) | 136 | (4.7%) |
| SAEs thought to be related to study | 0 | | 0 | |
| vaccine | | | | |
| AE with fatal outcome | 8 | (0.3%) | 12 | (0.4%) |
| AE with fatal outcome thought to be | 0 | | 0 | |
| related to study vaccine | | | | |
| AE leading to permanent stop | 10 | (0.3%) | 15 | (0.5%)* |

*For 2 placebo participants with a fatal AE, the AE is not yet indicated as leading to discontinuation in the AE database

It has thus been shown that the vaccine combination of the invention has an acceptable safety profile.

As described, this study is evaluating the vaccine regimen selected in the Phase 1/2a study VAC18193RSV1004, which consists of a mix of Ad26.RSV.preF (1×10¹¹ vp) and RSV preF protein (150 μg) (Ad26.RSV.preF/RSV preF protein), administered as a single injection. The primary analysis after the first RSV season has been completed and follow-up of participants through a second RSV season is ongoing.

This study thus has a recent revaccination cohort included at day 365 in which a total of approximately 240 participants received Ad26.RSV.preF/RSV preF protein on Day 365. Half of the participants in this revaccination cohort were taken from the active arm of the study in which these subjects received Ad26/protein preF RSV vaccine on Day 1 and the other half from the placebo arm. In this cohort the vaccine-induced immune responses following month 12 revaccination from Ad26/protein preF RSV vaccine will be examined following year 1 revaccination. Humoral immunogenicity will be assessed in this cohort from serum collected at 1 day, 14 days, 28 days, 3 months, 6 months and 12 months following first vaccination and month 12 revaccination. Recent data from this revaccination cohort showed that humoral immune responses (preF ELISA, postF ELISA and VNA_A2) were still significantly higher (approximately 4-fold) than baseline at both 14 days and 28 days post revaccination. At 15 days post revaccination, geometric mean VNA_A2 and pre-F ELISA titers increased less than 2-fold compared to prior to revaccination and remained approximately 2.5-2.7 fold lower as compared to geometric mean titers (GMTs) 15 days post first vaccination (FIG. 29 and FIG. 30). This data further confirms the month 12 revaccination humoral immunogenicity results from SR1004 cohort 3 (FIG. 26 and FIG. 27).

### Example 7: Phase 1/2a Study VAC18193RSV1004—Durability of Immune Responses and Immunogenicity Upon Revaccination

Durability of the vaccine-induced immune responses and immune responses after revaccination were evaluated in the ongoing Phase 1/2a study VAC18193RSV1004 in adult participants aged 60 years and older who are in stable health.

The study design includes 3 sequential cohorts: an initial safety cohort (Cohort 1 with a total of 64 participants) for the RSV preF protein containing vaccine regimen, a regimen selection cohort (Cohort 2 with a total of 288 participants), and an expanded safety cohort (Cohort 3 with a total of 315 participants).

The long-term durability of the humoral and cellular immune response after a single immunization is being evaluated in 2 groups of Cohort 2, which received Ad26.RSV.preF/RSV preF protein at a dose level of 1×10¹¹ vp/150 μg (Group 14) and 5×10¹⁰ vp/150 μg (Group 15). The kinetics of humoral and cellular immune responses is assessed in these groups by analyzing samples collected 14 days, 28 days, 56 days, 26 weeks, 12 months, 18 months, 24 months, 30 months, and 36 months post vaccination.

FIG. 25 shows the immunogenicity data from the Ad26.RSV.preF/RSV preF protein group that received Ad26.RSV.preF/RSV preF protein (1×10¹¹ vp/150 μg) (Group 14) up to 18 months post vaccination. Humoral immune responses assessed by pre-F ELISA and virus neutralization assay against RSV A2 (VNA A2) peaked around 15 days following initial vaccination (˜13-fold above baseline) and then decayed to reach a plateau at 1 year, remaining ˜4-fold above baseline levels up to 1.5 year, the latest timepoint analyzed. The cellular immune responses as measured by RSV F-specific interferon (IFN)γ enzyme-linked immunospot (ELISpot) had a similar kinetic.

In Cohort 3 (expanded safety cohort), immunogenicity after revaccination is being evaluated. A total of 270 participants have received Ad26.RSV.preF/RSV preF protein at 1×10¹¹ vp/150 μg (Ad26.RSV.preF/RSV preF protein) on Day 1. Half of the participants are to receive an additional vaccination at Month 12 and Month 24, whereas the other half will only receive an additional vaccination at Month 24 (see Table 1).

TABLE 1
Study Design VAC18193RSV1004: Expanded Cohort (Cohort 3)

| Group | N | Day 1 | Month 12 | Month 24 |
|---|---|---|---|---|
| 19 | 135 | Ad26.RSV.preF/ | Ad26.RSV.preF/ | Ad26.RSV.preF/ |
| | | RSV preF protein | RSV preF protein | RSV preF protein |
| | | mixture | mixture | mixture |
| | | 1 × 10¹¹ vp/ | 1 × 10¹¹ vp/ | 1 × 10¹¹ vp/ |
| | | 150 μg | 150 μg | 150 μg |
| 20 | 135 | Ad26.RSV.preF/ | Placebo | Ad26.RSV.preF/ |
| | | RSV preF protein | | RSV preF protein |
| | | mixture | | mixture |
| | | 1 × 10¹¹ vp/ | | 1 × 10¹¹ vp/ |
| | | 150 μg | | 150 μg* |
| 21 | 45 | Placebo | Placebo | Placebo |
| Total | 315 | | | |

N = number of participants; vp = virus particles.
*A protocol amendment to add this revaccination is currently under review.

With this study design, the durability of the vaccine-induced immune responses from Ad26/protein preF RSV vaccine will be examined in Cohort 3, both with yearly revaccination at Year 1 and 2, or with revaccination at Year 2. In addition, the kinetics of the cellular immune responses will be available in a subset (n=63) of these participants (2:2:1 randomization).
The kinetics of immune responses will be analyzed for 3 years in all participants. Of note, kinetics of the immune responses for 3 years without revaccination will be available for Group 14 in Cohort 2.
Recent data from Cohort 3 assessed the immune responses in the active vaccine groups with and without revaccination at Month 12 with data available up to 28 days post Month 12 revaccination (Day 393). At the time of revaccination at Month 12, humoral and cellular immune responses were still significantly higher (approximately 4-fold) than baseline. At 28 days after revaccination, geometric mean VNA A2 and pre-F ELISA titers increased 1.4- and 2.0-fold compared to prior to revaccination, respectively, to reach levels 4- to 5-fold higher than baseline but remained approximately 2-fold lower as compared to geometric mean titers (GMTs) 28 days post first vaccination (FIG. 26 and FIG. 27). Cellular immune responses as measured by IFNγ ELISpot were increased 2.5-fold 28 days after the Month 12 revaccination compared to prior to revaccination, reaching levels comparable to those 28 days after the first vaccination (FIG. 28, restricted to participants with Day 393 data). There was no correlation observed for Ad26 neutralizing antibodies measured prior to first vaccination or prior to revaccination at day 365 and post vaccination or revaccination induced immune responses (preF ELISA, postF ELISA, VNA_A2 and INFγ ELISPOT) respectively.

SEQUENCES

SEQ ID NO: 1 (RSV F protein A2 full length sequence)
MELLILKANAITTILTAVTFCFASGQNITEEFYQSTCSAVSKGYLSALRTGWYTSVITIE
LSNIKKNKCNGTDAKIKLIKQELDKYKNAVTELQLLMQSTPATNNRARRELPRFMN
YTLNNAKKTNVTLSKKRKRRFLGFLLGVGSAIASGVAVSKVLHLEGEVNKIKSALLS
TNKAVVSLSNGVSVLTSKVLDLKNYIDKQLLPIVNKQSCSISNIETVIEFQQKNNRLLE
ITREFSVNAGVTTPVSTYMLTNSELLSLINDMPITNDQKKLMSNNVQIVRQQSYSIMSI
IKEEVLAYVVQLPLYGVIDTPCWKLHTSPLCTTNTKEGSNICLTRTDRGWYCDNAGS
VSFFPQAETCKVQSNRVFCDTMNSLTLPSEVNLCNVDIFNPKYDCKIMTSKTDVSSSV
ITSLGAIVSCYGKTKCTASNKNRGIIKTFSNGCDYVSNKGVDTVSVGNTLYYVNKQE
GKSLYVKGEPIINFYDPLVFPSDEFDASISQVNEKINQSLAFIRKSDELLHNVNAVKST
TNIMITTIIIVIIVILLSLIAVGLLLYCKARSTPVTLSKDQLSGINNIAFSN
SEQ ID NO: 2 (Trimerization domain)
GYIPEAPRDGQAYVRKDGEWVLLSTFL
SEQ ID NO: 3 (Linker)
SAIG
SEQ ID NO: 4 (insert Ad26.preF)
ATGGAGCTGCTGATCCTGAAGGCCAACGCCATCACCACCATCCTGACCGCCGTGACCTTCTGCTTCGCCAGCGGCCAGAACATCACCGA
GGAATTCTACCAGAGCACCTGTAGCGCCGTGTCCAAGGGCTACCTGAGCGCCCTGAGAACCGGCTGGTACACCAGCGTGATCACCATCG
AGCTGAGCAACATCAAAGAAATCAAGTGCAACGGCACCGACGCCAAAGTGAAGCTGATCAAGCAGGAACTGGACAAGTACAAGAACG
CCGTGACCGAGCTGCAGCTGCTGATGCAGAGCACCCCCGCCACCAACAACCGGGCCAGACGCGAGCTGCCCCGGTTCATGAACTACAC
CCTGAACAACGCCAAAAAGACCAACGTGACCCTGAGCAAGAAGCGGAAGCGGCGGTTCCTGGGCTTCCTGCTGGGCGTGGGCTCTGCC
ATTGCTAGCGGAGTGGCCGTGTCTAAAGTGCTGCACCTGGAAGGCGAAGTGAACAAGATCAAGAGCGCCCTGCTGAGCACCAACAAGG
CCGTGGTGTCCCTGAGCAACGGCGTGTCCGTGCTGACCAGCAAGGTGCTGGATCTGAAGAACTACATCGACAAGCAGCTGCTGCCCATC
GTGAACAAGCAGAGCTGCAGCATCCCCAACATCGAGACAGTGATCGAGTTCCAGCAGAAGAACAACCGGCTGCTGGAAATCACCCGCG
AGTTCAGCGTGAACGCTGGCGTGACCACCCCCGTGTCCACCTACATGCTGACCAACAGCGAGCTGCTGTCCCTGATCAATGACATGCCC
ATCACCAACGACCAGAAAAAGCTGATGAGCAACAACGTGCAGATCGTGCGGCAGCAGAGCTACTCCATCATGTCCATCATCAAAGAAG
AGGTGCTGGCCTACGTGGTGCAGCTGCCCCTGTACGGCGTGATCGACACCCCCTGCTGGAAGCTGCACACCAGCCCCCTGTGCACCACC
AACACCAAAGAGGGCAGCAACATCTGCCTGACCCGGACCGACCGGGGCTGGTACTGCGATAATGCCGGCTCCGTGTCATTCTTTCCACA
AGCCGAGACATGCAAGGTGCAGAGCAACCGGGTGTTCTGCGACACCATGAACAGCCTGACCCTGCCCTCCGAAGTGAACCTGTGCAAC
GTGGACATCTTCAACCCTAAGTACGACTGCAAGATCATGACCTCCAAGACCGACGTGTCCAGCTCCGTGATCACCTCCCTGGGCGCCATC
GTGTCCTGCTACGGCAAGACCAAGTGCACCGCCAGCAACAAGAACCGGGGCATCATCAAGACCTTCAGCAACGGCTGCGACTACGTGT
CCAACAAGGGGGTGGACACCGTGTCCGTGGGCAACACCCTGTACTACGTGAACAAACAGGAAGGCAAGAGCCTGTACGTGAAGGGCG
AGCCCATCATCAACTTCTACGACCCCCTGGTGTTCCCCAGCAACGAGTTCGACGCCAGCATCAGCCAGGTCAACGAGAAGATCAACCAG
AGCCTGGCCTTCATCAGAAAGAGCGACGAGCTGCTGCACAATGTGAATGCCGTGAAGTCCACCACCAATATCATGATCACCACAATCAT
CATCGTGATCATTGTGATCCTGCTGAGCCTGATCGCCGTGGGCCTGCTGCTGTACTGCAAGGCCAGATCCACCCCTGTGACCCTGTCCAA
GGACCAGCTGAGCGGCATCAACAATATCGCCTTCTCCAACTGATAA
SEQ ID NO: 5 RSV F protein encoded by Ad26.preF
MELLILKANAITTILTAVTFCFASGQNITEEFYQSTCSAVSKGYLSALRTGWYTSVITIELSNIKEIKCNGTDAKVKLIKQELDKYKNA
VTELQLLMQSTPATNNRARRELPRFMNYTLNNAKKTNVTLSKKRKRRFLGFLLGVGSAIASGVAVSKVLHLEGEVNKIKSALLST
NKAVVSLSNGVSVLTSKVLDLKNYIDKQLLPIVNKQSCSIPNIETVIEFQQKNNRLLEITREFSVNAGVTTPVSTYMLTNSELLSLIN
DMPITNDQKKLMSNNVQIVRQQSYSIMSIIKEEVLAYVVQLPLYGVIDTPCWKLHTSPLCTTNTKEGSNICLTRTDRGWYCDNA
GSVSFFPQAETCKVQSNRVFCDTMNSLTLPSEVNLCNVDIFNPKYDCKIMTSKTDVSSSVITSLGAIVSCYGKTKCTASNKNRGII
KTFSNGCDYVSNKGVDTVSVGNTLYYVNKQEGKSLYVKGEPIINFYDPLVFPSNEFDASISQVNEKINQSLAFIRKSDELLHNVNA
VKSTTNIMITTIIIVIIVILLSLIAVGLLLYCKARSTPVTLSKDQLSGINNIAFSN**
SEQ ID NO: 6 soluble RSV preF protein (precursor, i.e. not processed)
MELLILKANAITTILTAVTFCFASGQNITEEFYQSTCSAVSKGYLSALRTGWYTSVITIELSNIKEIKCNG
TDAKVKLIKQELDKYKNAVTELQLLMQSTPATNNRARRELPRFMNYTLNNAKKTNVTLSKKRKRRFL
GFLLGVGSAIASGVAVSKVLHLEGEVNKIKSALLSTNKAVVSLSNGVSVLTSKVLDLKNYIDKQLLPIV
NKQSCSIPNIETVIEFQQKNNRLLEITREFSVNAGVTTPVSTYMLTNSELLSLINDMPITNDQKKLMSNN
VQIVRQQSYSIMSIIKEEVLAYVVQLPLYGVIDTPCWKLHTSPLCTTNTKEGSNICLTRTDRGWYCDNA
GSVSFFPQAETCKVQSNRVFCDTMNSLTLPSEVNLCNVDIFNPKYDCKIMTSKTDVSSSVITSLGAIVSC
YGKTKCTASNKNRGIIKTFSNGCDYVSNKGVDTVSVGNTLYYVNKQEGKSLYVKGEPIINFYDPLVFP
SNEFDASISQVNEKINQSLAFIRKSDELLSAIGGYIPEAPRDGQAYVRKDGEWVLLSTFL
Signal peptide: double underlined
Antigen: no underline
SEQ ID NO: 7 soluble RSV preF protein processed
QNITEEFYQSTCSAVSKGYLSALRTGWYTSVITIELSNIKEIKCNGTDAKVKLIKQELDKY
KNAVTELQLLMQSTPATNNRARRFLGFLLGVGSAIASGVAVSKVLHLEGEVNKIKSALL
STNKAVVSLSNGVSVLTSKVLDLKNYIDKQLLPIVNKQSCSIPNIETVIEFQQKNNRLLEIT
REFSVNAGVTTPVSTYMLTNSELLSLINDMPITNDQKKLMSNNVQIVRQQSYSIMSIIKEE
VLAYVVQLPLYGVIDTPCWKLHTSPLCTTNTKEGSNICLTRTDRGWYCDNAGSVSFFPQ
AETCKVQSNRVFCDTMNSLTLPSEVNLCNVDIFNPKYDCKIMTSKTDVSSSVITSLGAIV
SCYGKTKCTASNKNRGIIKTFSNGCDYVSNKGVDTVSVGNTLYYVNKQEGKSLYVKGE
PIINFYDPLVFPSNEFDASISQVNEKINQSLAFIRKSDELLSAIGGYIPEAPRDGQAYVRKD GEWVLLSTFL
SEQ ID NO: 8 nucleotide sequence coding for RSV preF protein
atggaactgctgatcctgaaggccaacgccatcaccaccatcctgaccgccgtgaccttctgctttgccagcggccagaacatcaccgaggagttctacca
gagcacctgtagcgccgtgtccaagggctacctgagcgccctgagaaccggctggtacaccagcgtgatcaccatcgagctgagcaacatcaaagaaat
caagtgcaacggcaccgacgccaaagtgaagctgatcaagcaggaactggacaagtacaagaatgccgtgaccgaactgcagctgctgatgcagagca
cccccgccaccaacaaccgggccagaagagaactgcccagattcatgaactacaccctgaacaacgccaaaaagaccaacgtgaccctgagcaagaa
gcggaagcggcggttcctgggctttctgctgggagtgggaagcgccattgctagcggagtggccgtgtctaaggtgctgcacctggaaggcgaagtgaa
caagatcaagtccgccctgctgagcaccaacaaggccgtggtgtctctgagcaacggcgtgtccgtgctgaccagcaaggtgctggatctgaagaactac
atcgacaaacagctgctgcccatcgtgaacaagcagagctgcagcatccccaacatcgagacagtgatcgagttccagcagaagaacaaccggctgctg
gaaatcacccgcgagttcagcgtgaacgctggcgtgaccacccccgtgtccacctacatgctgaccaacagcgagctgctgtccctgatcaacgacatgc
ccatcaccaacgaccagaaaaagctgatgagcaacaacgtgcagatcgtgcggcagcagagctactccatcatgagcattatcaaagaagaggtgctgg
cctacgtggtgcagctgcctctgtacggcgtgatcgacaccccctgctggaagctgcacaccagccctctgtgcaccaccaacaccaaagagggcagca
acatctgcctgacccggaccgacagaggctggtactgcgataatgccggctccgtctcattctttccacaagccgagacatgcaaggtgcagagcaaccg
ggtgttctgcgacaccatgaacagcctgaccctgccctccgaagtgaatctgtgcaacgtggacatcttcaaccctaagtacgactgcaagatcatgacctc
caagaccgacgtgtccagctccgtgatcacaagcctgggcgccatcgtgtcctgctacggcaagaccaagtgcaccgccagcaacaagaaccggggca
tcatcaagaccttcagcaacggctgcgactacgtgtccaacaagggggtggacaccgtgtctgtgggcaacaccctgtactacgtgaacaaacaggaagg
caagagcctgtacgtgaagggcgagcccatcatcaacttctacgaccccctggtgttccccagcaacgagttcgacgccagcatcagccaagtgaacgag
aagatcaaccagagcctggccttcatcagaaagtccgatgagctgctgagcgccatcggcggctacatccctgaggcccctagagatggccaggcctatg
tgcggaaggacggcgaatgggtgctgctgtctaccttcctgtga
Signal peptide: double underlined
Antigen: no underline
SEQ ID NO: 9 (5′ terminal nucleotides of recombinant adenovectors)
CTATCTAT
SEQ ID NO: 10 (5′ terminal nucleotides of original adenovectors)
CATCATCA

1. A method of inducing a protective immune response against respiratory syncytial virus (RSV) infection in a human subject in need thereof, comprising administering to the subject a combination comprising:
(a) an effective amount of a first immunogenic component, comprising an adenoviral vector comprising a nucleic acid encoding an RSV F protein that is stabilized in a pre-fusion conformation, preferably the effective amount of the first immunogenic component comprises about 1×10¹⁰ to about 1×10¹² viral particles of the adenoviral vector per dose; and
(b) an effective amount of a second immunogenic component, comprising a soluble RSV F protein that is stabilized in a pre-fusion conformation, preferably the effective amount of the second immunogenic component comprises about 30 ug to about 300 ug of the RSV F protein per dose,
preferably (a) and (b) are co-administered.

2. The method of claim 1, wherein the adenoviral vector is replication-incompetent and has a deletion in at least one of the adenoviral early region 1 (E1 region) and the early region 3 (E3 region).

3. The method of claim 2, wherein the adenoviral vector is a replication-incompetent Ad26 adenoviral vector having a deletion of the E1 region and the E3 region.

4. The method of claim 2, wherein the adenoviral vector is a replication-incompetent Ad35 adenoviral vector having a deletion of the E1 region and the E3 region.

5. The method of any one of claims 1-4, wherein the recombinant RSV F protein encoded by the adenoviral vector has the amino acid sequence of SEQ ID NO: 5.

6. The method of any one of claims 1-5, wherein the nucleic acid encoding the RSV F protein comprises the polynucleotide sequence of SEQ ID NO: 4.

7. The method of any of claims 1-6, wherein the soluble RSV F protein that is stabilized in a pre-fusion conformation has the amino acid sequence of SEQ ID NO: 6 or 7.

8. The method of any one of claims 1-7, wherein the soluble RSV F protein that is stabilized in a pre-fusion conformation is encoded by a nucleic acid having the nucleotide sequence of SEQ ID NO: 8.

9. The method of any one of claims 1-8, wherein the effective amount of the first immunogenic component comprises about 1×10¹¹ viral particles of the adenoviral vector per dose.

10. The method of any one of claims 1-9, wherein the effective amount of the second immunogenic component comprises about 150 ug of the RSV F protein per dose.

11. The method of any one of claims 1-10, further comprising administering to the subject:
(c) an effective amount of the first immunogenic component, preferably the effective amount comprises about 1×10¹⁰ to about 1×10¹² viral particles of the adenoviral vector per dose; and
(d) an effective amount of the second immunogenic component, preferably the effective amount comprises about 30 ug to about 300 ug of the RSV F protein per dose.
after the initial administration.

12. The method of any one of claims 1-11, wherein the subject is susceptible to the RSV infection.

13. The method of any one of claims 1-12, wherein the subject is ≥60 years old, preferably is ≥65 years old.

14. The method of any one of claims 1-13, wherein the protective immune response is characterized by the prevention or reduction of reverse transcriptase polymerase chain reaction (RT PCR)-confirmed RSV-mediated lower respiratory tract disease (LRTD).

15. The method of any one of claims 1-14, wherein the protective immune response is characterized by an absent or reduced RSV viral load in the nasal track and/or lungs of the subject upon exposure to RSV.

16. The method of any one of claims 1-15, wherein the protective immune response is characterized by an absent or reduced RSV clinical symptom in the subject upon exposure to RSV.

17. The method of any one of claims 1-16, wherein the protective immune response is characterized by the presence of neutralizing antibodies to RSV and/or protective immunity against RSV, preferably detected between at least 15 to 169 days after administration of the immunogenic components.

18. A method of safely preventing infection and/or replication of RSV in a human subject in need thereof, comprising prophylactically administering intramuscularly to the subject a combination comprising:
(a) an effective amount of a first immunogenic component, comprising about 1×10¹⁰ to about 1×10¹² viral particles per dose of an adenoviral vector comprising a nucleic acid encoding an RSV F protein having the amino acid sequence of SEQ ID NO: 5, wherein the adenoviral vector is replication-incompetent; and
(b) an effective amount of a second immunogenic component, comprising about 30 ug to about 300 ug per dose of an RSV F protein having an amino acid sequence of SEQ ID NO; 6 or 7,
wherein (a) and (b) are co-administered.

19. The method of claim 18, wherein the adenoviral vector is a replication-incompetent Ad26 adenoviral vector having a deletion of the E1 region and the E3 region.

20. The method of claim 18 or 19, wherein the effective amount of the first immunogenic component comprises about 1×10¹¹ viral particles of the adenoviral vector per dose.

21. The method of any one of claims 18-20, wherein the effective amount of the second immunogenic component comprises about 150 ug of the RSV F protein per dose.

22. The method of any one of claims 18-21, further comprising administering to the subject:
(c) an effective amount of the first immunogenic component comprising about 1×10¹⁰ to about 1×10¹² viral particles of the adenoviral vector per dose; and
(d) an effective amount of the second immunogenic component comprising about 30 ug to about 300 ug of the RSV F protein per dose.
after the initial administration.

23. The method of any one of claims 18-22, wherein the subject is susceptible to the RSV infection.

24. The method of any one of claims 18-23, wherein the subject is ≥60 years old.

25. The method of any one of claims 18-24, wherein the prevented infection and/or replication of RSV is characterized by the prevention or reduction of reverse transcriptase polymerase chain reaction (RT PCR)-confirmed RSV-mediated lower respiratory tract disease (LRTD).

26. The method of any one of claims 18-25, wherein the prevented infection and/or replication of RSV is characterized by an absent or reduced RSV viral load in the nasal track and/or lungs of the subject.

27. The method of any one of claims 18-26, wherein the prevented infection and/or replication of RSV is characterized by an absent or reduced RSV clinical symptom in the subject upon exposure to RSV.

28. The method of any one of claims 18-27, wherein the protective immune response is characterized by the presence of neutralizing antibodies to RSV and/or protective immunity against RSV, preferably detected at least between 15 to 169 days after administration of the immunogenic components.

29. An immunogenic combination, containing (a) a first immunogenic component comprising an adenoviral vector comprising a nucleic acid encoding an RSV F protein that is stabilized in a pre-fusion conformation, and (b) a second immunogenic component comprising a soluble RSV F protein that is stabilized in a pre-fusion conformation, for simultaneous, separate or sequential use in inducing a protective immune response against RSV infection in a human subject in need thereof, preferably, the first and second immunogen components are co-administered, more preferably, the first immunogen component is administered at an effective amount of about 1×10¹⁰ to about 1×10¹² viral particles of the adenoviral vector per dose, and the second immunogenic component is administered at an effective amount of about 30 ug to about 300 ug of the RSV F protein per dose.


SEQUENCE LISTING (8 sequences)

SEQ ID NO: 1 - protein, 574 aa, respiratory syncytial virus
MELLILKANAITTILTAVTFCFASGQNITEEFYQSTCSAVSKGYLSALRTGWYTSVITIELSNIKKNKCNGTDAKIKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 2 - protein, 27 aa, Artificial Sequence
  Trimerization domain
GYIPEAPRDGQAYVRKDGEWVLLSTFL

SEQ ID NO: 3 - protein, 4 aa, Artificial Sequence
  Linker
SAIG

SEQ ID NO: 4 - DNA, 1728 bp, Artificial Sequence
  insert Ad26.preF
atggagctgctgatcctgaaggccaacgccatcaccaccatcctgaccgccgtgaccttctgcttcgccagcggccagaacatcaccgaggaattctaccagagcacctgtagcgccgtgtccaagggctacctgagcgccctgagaaccggctggtacaccagcgtgatcaccatcgagctgagcaacatcaaagaaatcaagtgcaacggcaccgacgccaaagtgaagctgatcaagcaggaactggacaagtacaagaacgccgtgaccgagctgcagctgctgatgcagagcacccccgccaccaacaaccgggccagacgcgagctgccccggttcatgaactacaccctgaacaacgccaaaaagaccaacgtgaccctgagcaagaagcggaagcggcggttcctgggcttcctgctgggcgtgggctctgccattgctagcggagtggccgtgtctaaagtgctgcacctggaaggcgaagtgaacaagatcaagagcgccctgctgagcaccaacaaggccgtggtgtccctgagcaacggcgtgtccgtgctgaccagcaaggtgctggatctgaagaactacatcgacaagcagctgctgcccatcgtgaacaagcagagctgcagcatccccaacatcgagacagtgatcgagttccagcagaagaacaaccggctgctggaaatcacccgcgagttcagcgtgaacgctggcgtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgtccatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggctccgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcaacgagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcattgtgatcctgctgagcctgatcgccgtgggcctgctgctgtactgcaaggccagatccacccctgtgaccctgtccaaggaccagctgagcggcatcaacaatatcgccttctccaactgataa

SEQ ID NO: 5 - protein, 574 aa, Artificial Sequence
  protein encoded by Ad26.preF
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 6 - protein, 544 aa, Artificial Sequence
  soluble RSV preF protein (precursor)
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLSAIGGYIPEAPRDGQAYVRKDGEWVLLSTFL

SEQ ID NO: 7 - protein, 492 aa, Artificial Sequence
  soluble RSV preF protein processed
QNITEEFYQSTCSAVSKGYLSALRTGWYTSVITIELSNIKEIKCNGTDAKVKLIKQELDKYKNAVTELQLLMQSTPATNNRARRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLSAIGGYIPEAPRDGQAYVRKDGEWVLLSTFL

SEQ ID NO: 8 - DNA, 1635 bp, Artificial Sequence
  nucleotide sequence coding for RSV preF protein
atggaactgctgatcctgaaggccaacgccatcaccaccatcctgaccgccgtgaccttctgctttgccagcggccagaacatcaccgaggagttctaccagagcacctgtagcgccgtgtccaagggctacctgagcgccctgagaaccggctggtacaccagcgtgatcaccatcgagctgagcaacatcaaagaaatcaagtgcaacggcaccgacgccaaagtgaagctgatcaagcaggaactggacaagtacaagaatgccgtgaccgaactgcagctgctgatgcagagcacccccgccaccaacaaccgggccagaagagaactgcccagattcatgaactacaccctgaacaacgccaaaaagaccaacgtgaccctgagcaagaagcggaagcggcggttcctgggctttctgctgggagtgggaagcgccattgctagcggagtggccgtgtctaaggtgctgcacctggaaggcgaagtgaacaagatcaagtccgccctgctgagcaccaacaaggccgtggtgtctctgagcaacggcgtgtccgtgctgaccagcaaggtgctggatctgaagaactacatcgacaaacagctgctgcccatcgtgaacaagcagagctgcagcatccccaacatcgagacagtgatcgagttccagcagaagaacaaccggctgctggaaatcacccgcgagttcagcgtgaacgctggcgtgaccacccccgtgtccacctacatgctgaccaacagcgagctgctgtccctgatcaacgacatgcccatcaccaacgaccagaaaaagctgatgagcaacaacgtgcagatcgtgcggcagcagagctactccatcatgagcattatcaaagaagaggtgctggcctacgtggtgcagctgcctctgtacggcgtgatcgacaccccctgctggaagctgcacaccagccctctgtgcaccaccaacaccaaagagggcagcaacatctgcctgacccggaccgacagaggctggtactgcgataatgccggctccgtctcattctttccacaagccgagacatgcaaggtgcagagcaaccgggtgttctgcgacaccatgaacagcctgaccctgccctccgaagtgaatctgtgcaacgtggacatcttcaaccctaagtacgactgcaagatcatgacctccaagaccgacgtgtccagctccgtgatcacaagcctgggcgccatcgtgtcctgctacggcaagaccaagtgcaccgccagcaacaagaaccggggcatcatcaagaccttcagcaacggctgcgactacgtgtccaacaagggggtggacaccgtgtctgtgggcaacaccctgtactacgtgaacaaacaggaaggcaagagcctgtacgtgaagggcgagcccatcatcaacttctacgaccccctggtgttccccagcaacgagttcgacgccagcatcagccaagtgaacgagaagatcaaccagagcctggccttcatcagaaagtccgatgagctgctgagcgccatcggcggctacatccctgaggcccctagagatggccaggcctatgtgcggaaggacggcgaatgggtgctgctgtctaccttcctgtga